(19) United States
(12) Patent Application Publication
(10) Pub. No.: US 2020/0188525 A1
(43) Pub. Date: Jun. 18, 2020

(54) ANTI-EGFR ANTIBODY DRUG CONJUGATES (ADC) AND USES THEREOF

(71) Applicant: AbbVie Inc., North Chicago, IL (US)

(72) Inventors: Edward Reilly, Libertyville, IL (US); Mark Anderson, Grayslake, IL (US)

(21) Appl. No.: 16/643,527

(22) PCT Filed: Sep. 4, 2018
(86) PCT No.: PCT/US2018/049412

§ 371 Date: Feb. 29, 2020

Related U.S. Application Data
(60) Provisional application No. 62/553,837, filed on Sep. 2, 2017.

(51) Int. Cl. A61K 47/68; C07K 16/28

(52) U.S. Cl. CPC A61K 47/6803; A61K 47/6849; C07K 16/2863; A61K 2039/505; C07K 2317/94; C07K 2317/565; C07K 2317/24

(57) ABSTRACT

Provided herein are antibody-drug conjugates that bind EGFR, in particular human EGFR, their methods of making, and their uses to treat patients having cancer.

### CROSS-REFERENCE TO RELATED APPLICATIONS

This application claims the benefit of U.S. Provisional Application Ser. No. 62/553,837, filed Sep. 2, 2017, the disclosure of which is hereby incorporated by reference in its entirety.

### FIELD

The present disclosure pertains to, among other things, human epidermal growth factor receptor (EGFR, also known as HER-1 or Erb-B1) antibody drug conjugates (ADCs), compositions comprising such ADCs, methods of making the ADCs, and uses thereof.

### BACKGROUND

Cancer therapies comprise a wide range of therapeutic approaches, including surgery, radiation, and chemotherapy. While the often complementary approaches allow a broad selection to be available to the medical practitioner to treat the cancer, existing therapeutics suffer from a number of disadvantages, such as a lack of selectivity of targeting cancer cells over normal, healthy cells, and the development of resistance by the cancer to the treatment.

Recent approaches to treating cancer based on targeted therapeutics, such as antibodies, have led to chemotherapeutic regimens with fewer side effects as compared to non-targeted therapies such as radiation treatment. One effective approach for enhancing the anti-tumor-potency of antibodies involves linking cytotoxic drugs or toxins to monoclonal antibodies that are capable of being internalized by a target cell. These agents are termed antibody-drug conjugates (ADCs). Upon administration to a patient, ADCs bind to target cells via their antibody portions and become internalized, allowing the drugs or toxins to exert their effect (see, e.g., U.S. Patent Appl. Publ. Nos. US2005/0180972 and US2005/0123536).

The human epidermal growth factor receptor is a 170 kDa transmembrane receptor encoded by the c-erbB protooncogene, and exhibits intrinsic tyrosine kinase activity (Modjtahedi et al., Br. J. Cancer 73:228-235 (1996); Herbst and Shin, Cancer 94:1593-1611 (2002)). SwissProt database entry P00533 provides the sequence of human EGFR. EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay et al., Ann. Oncology 14:1346-1363 (2003); Tsao and Herbst, Signal 4:4-9 (2003); Herbst and Shin, Cancer 94:1593-1611 (2002); Modjtahedi et al., Br. J. Cancer 73:228-235 (1996)).

Known ligands of EGFR include EGF, TGFA/TGF-alpha, amphiregulin, epigen/EPGN, BTC/betacellulin, epiregulin/EREG and HBEGF/heparin-binding EGF. Ligand binding by EGFR triggers receptor homo- and/or heterodimerization and autophosphorylation of key cytoplasmic residues. The phosphorylated EGFR recruits adapter proteins like GRB2 which in turn activate complex downstream signaling cascades, including at least the following major downstream signaling cascades: the RAS-RAF-MEK-ERK., PI3 kinase-AKT, PLCgamma-PKC, and STATS modules. This autophosphorylation also elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK. pathways, leading to cell proliferation. Ligand binding by EGFR may also activate the NF-kappa-B signaling cascade. Ligand binding also directly phosphorylates other proteins like RGS16, activating its GTPase activity and potentially coupling the EGF receptor signaling to G protein-coupled receptor signaling. Ligand binding also phosphorylates MUC1 and increases its interaction with SRC and CTNNB 1/beta-catenin.

Overexpression of EGFR has been reported in numerous human malignant conditions, including cancers of the bladder, brain, head and neck, pancreas, lung, breast, ovary, colon, prostate, and kidney. (Atalay et al., Ann. Oncology 14:1346-1363 (2003); Herbst and Shin, Cancer 94:1593-1611 (2002); and Modjtahedi et al., Br. J. Cancer 73:228-235 (1996)). In many of these conditions, the overexpression of EGFR correlates or is associated with poor prognosis of the patients. (Herbst and Shin, Cancer 94:1593-1611 (2002); and Modjtahedi et al., Br. J. Cancer 73:228-235 (1996)). EGFR is also expressed in the cells of normal tissues, particularly the epithelial tissues of the skin, liver, and gastrointestinal tract, although at generally lower levels than in malignant cells (Herbst and Shin, Cancer 94:1593-1611 (2002)).

A significant proportion of tumors containing amplifications of the EGFR gene (i.e., multiple copies of the EGFR gene) also co-express a truncated version of the receptor (Wikstrand et al. (1998) J. Neurovirol. 4, 148-158) known as de2-7 EGFR, ΔEGFR, EGFRvIII, or Δ2-7 (terms used interchangeably herein) (Olapade-Olaopa et al. (2000) Br. J. Cancer. 82, 186-94). The rearrangement seen in the de2-7 EGFR results in an in-frame mature mRNA lacking 801 nucleotides spanning exons 2-7 (Wong et al. (1992) Proc. Natl. Acad. Sci. U.S.A. 89, 2965-9; Yamazaki et al. (1990) Jpn. J. Cancer Res. 81, 773-9; Yamazaki et al. (1988) Mol. Cell. Biol. 8, 1816-20; and Sugawa et al. (1990) Proc. Natl. Acad. Sci. U.S.A. 87, 8602-6). The corresponding EGFR protein has a 267 amino acid deletion comprising residues 6-273 of the extracellular domain and a novel glycine residue at the fusion junction (Sugawa et al., 1990). This deletion, together with the insertion of a glycine residue, produces a unique junctional peptide at the deletion interface (Sugawa et al., 1990).

EGFRvIII has been reported in a number of tumor types including glioma, breast, lung, ovarian and prostate (Wikstrand et al. (1997) Cancer Res. 57, 4130-40; Olapade-Olaopa et al. (2000) Br. J. Cancer. 82, 186-94; Wikstrand, et al. (1995) Cancer Res. 55, 3140-8; Garcia de Palazzo et al. (1993) Cancer Res. 53, 3217-20). While this truncated receptor does not bind ligand, it possesses low constitutive activity and imparts a significant growth advantage to glioma cells grown as tumor xenografts in nude mice (Nishikawa et al. (1994) Proc. Natl. Acad. Sci. U.S.A. 91, 7727-31) and is able to transform NIH3T3 cells (Batra et al. (1995) Cell Growth Differ. 6, 1251-9) and MCF-7 cells. The cellular mechanisms utilized by the de2-7 EGFR in glioma cells are not fully defined but are reported to include a decrease in apoptosis (Nagane et al. (1996) Cancer Res. 56, 5079-86) and a small enhancement of proliferation (Nagane et al., 1996). As expression of this truncated receptor is restricted to tumor cells it represents a highly specific target for antibody therapy.

Accordingly, there remains a need in the art for anti-EGFR antibodies and ADCs that can be used for therapeutic purposes in the treatment of cancer.

### SUMMARY

The present disclosure provides antibody-drug conjugates (ADCs) comprising a cytotoxic or cytostatic agent linked to an anti-EGFR antibody by way of a linker, compositions comprising the ADCs, methods of making the ADCs, and methods of treating a cancer comprising administering the ADCs to a subject having cancer. As described in more detail in the Examples, and while not intending to be bound by any particular theory of operation, the data included herein demonstrate that anti-EGFR ADCs comprising specific linkers and specific cytotoxic and/or cytostatic agents (i.e., a pyrrolobenzodiazepine (PBD) dimer), exert potent anti-tumor activities. Moreover, the anti-EGFR ADCs of the present disclosure are characterized by a fixed low drug loading, which surprisingly provides a highly efficacious ADC.

Accordingly, in embodiments, the present disclosure provides ADCs that specifically bind EGFR, and in particular human EGFR (hEGFR).

In embodiments, the present disclosure provides an antibody drug conjugate (ADC) comprising a cytotoxic and/or cytostatic agent linked to an antibody by way of a linker, wherein the ADC is a compound according to the structural formula (I):

[D-L-XY]n-Ab (I),

or a salt thereof, where D comprises a pyrrolobenzodiazepine (PBD) dimer, L is a linker, and Ab is an anti-human epidermal growth factor receptor antibody. In embodiments, the anti-EGFR Ab comprises (i) a heavy chain CDRH1 domain comprising the amino acid sequence set forth in SEQ ID NO: 3; a heavy chain CDRH2 domain comprising the amino acid sequence set forth in SEQ ID NO: 4, and a heavy chain CDRH3 domain comprising the amino acid sequence set forth in SEQ ID NO: 5; (ii) a light chain CDRL1 domain comprising the amino acid sequence set forth in SEQ ID NO: 8; a light chain CDRL2 domain comprising the amino acid sequence set forth in SEQ ID NO: 9; a light chain CDRL3 domain comprising the amino acid sequence set forth in SEQ ID NO: 10; and (iii) a mutation comprising S239C in a heavy chain constant region, wherein the numbering is in accordance with Kabat. XY represents a covalent linkage linking linker L to antibody Ab through the S239C mutation. In embodiments, n is any integer. In embodiments, n is 2. In embodiments, the antibody Ab has a heavy chain variable region comprising the amino acid sequence of SEQ ID NO: 2, and a light chain variable region comprising the amino acid sequence of SEQ ID NO: 7. In embodiments, the antibody Ab has a heavy chain comprising the amino acid sequence of SEQ ID NO: 1, and a light chain comprising the amino acid sequence of SEQ ID NO: 6. In embodiments, XY is a maleimide-sulfhydryl linkage. In embodiments, L comprises the linker as described in Formula III, IV, V, VI, VII, VIII, or IX. For example, in embodiments, L comprises the linker as described in Formula IX. In embodiments, the linker is a maleimidocaproyl-Valine-Alanine (mc-Val-Ala) linker. IN embodiments, the anti-EGFR antibody comprises an IgG1 isotype. In certain embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure provides an antibody-drug conjugate (ADC) comprising a cytotoxic and/or cytostatic agent linked to an antibody by way of a linker, wherein the antibody drug conjugate is a compound according to structural Formula (I)

[D-L-XY]n-Ab (I),

or a salt thereof, where D comprises a pyrrolobenzodiazepine (PBD) dimer; L is a linker; Ab is an anti-EGFR antibody comprising (i) a heavy chain variable region comprising SEQ ID NO:2, (ii) a light chain variable region comprising SEQ ID NO: 7; and (iii) a mutation comprising S239C in a heavy chain constant region, wherein the numbering is in accordance with Kabat; XY represents a covalent linkage linking linker L to antibody Ab; and n is any integer. In embodiments, n is 2 or 4. In embodiments, n is 2. In embodiments, XY is a linkage formed with a sulfhydryl group on antibody Ab. In embodiments, XY is a maleimide-sulfhydryl linkage. In embodiments, L comprises the linker as described in Formula III, IV, V, VI, VII, VIII, or IX. In embodiments, L comprises the linker as described in Formula IX. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure provides an antibody drug conjugate comprising a cytotoxic and/or cytostatic agent linked to an antibody by way of a linker, wherein the antibody drug conjugate is a compound according to structural formula (I):

[D-L-XY]n-Ab (I),

or a salt thereof, where D comprises a pyrrolobenzodiazepine (PBD) dimer; L is a linker; Ab is an anti-EGFR antibody comprising (i) a heavy chain comprising the amino acid sequence as set forth in SEQ ID NO: 1, (ii) a light chain comprising the amino acid sequence set forth in SEQ ID NO: 6; XY represents a covalent linkage linking linker L to antibody Ab; and n is any integer. In embodiments, n is 2 or 4. In embodiments, n is 2. In embodiments, XY is a linkage formed with a sulfhydryl group on antibody Ab. In embodiments, XY is a maleimide-sulfhydryl linkage. In embodiments, L comprises the linker as described in Formula III, IV, V, VI, VII, VIII, or IX. In embodiments, L comprises the linker as described in Formula IX. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure features an ADC comprising the structure of Formula (X):

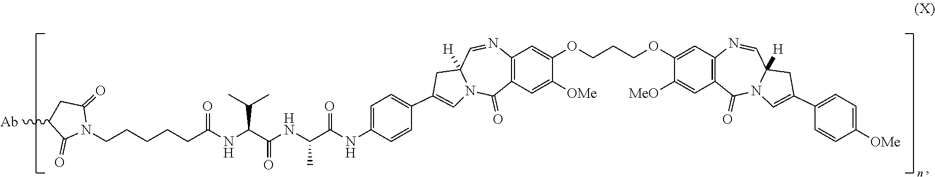

or a salt thereof, wherein Ab comprises an anti-EGFR antibody comprising (i) a heavy chain variable region comprising a CDRH1 sequence comprising SEQ ID NO: 3, a CDRH2 sequence comprising SEQ ID NO: 4, and a CDRH3 sequence comprising SEQ ID NO: 5; (ii) a light chain variable region comprising a CDRL1 sequence comprising SEQ ID NO: 8, a CDRL2 sequence comprising SEQ ID NO: 9, and a CDRL3 sequence comprising SEQ ID NO: 10; (iii) a mutation comprising S239C in a heavy chain constant region, wherein the numbering is in accordance with Kabat; wherein n is 2. In embodiments, the heavy chain variable region comprises SEQ ID NO: 2, and the light chain variable region comprises SEQ ID NO: 7. In embodiments, the ADC comprises a full heavy chain comprising SEQ ID NO: 1, and a full light chain comprising SEQ ID NO: 6. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure features an ADC comprising the structure of Formula (X):

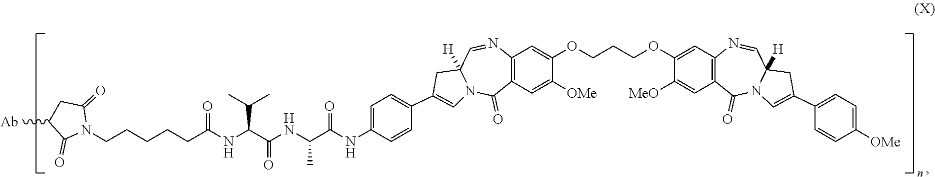

or a salt thereof, wherein Ab comprises an anti-EGFR antibody comprising (i) a heavy chain variable region comprising SEQ ID NO: 2; (ii) a light chain variable region comprising SEQ ID NO: 7; (iii) a mutation comprising S239C in a heavy chain constant region, wherein the numbering is in accordance with Kabat; wherein n is 2. In embodiments, the heavy chain variable region comprises SEQ ID NO: 2, and the light chain variable region comprises SEQ ID NO: 7. In embodiments, the ADC comprises a full heavy chain comprising SEQ ID NO: 1, and a full light chain comprising SEQ ID NO: 6. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure features an ADC comprising the structure of Formula (X):

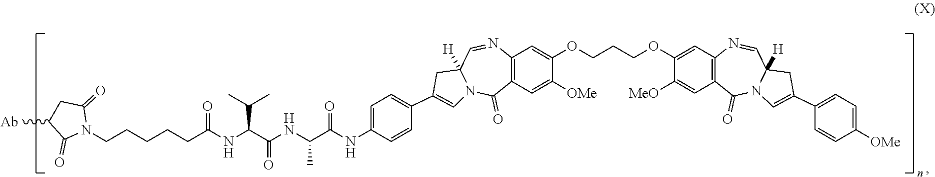

or a salt thereof, wherein Ab comprises an anti-EGFR antibody comprising (i) a heavy chain comprising SEQ ID NO: 1; (ii) a light chain comprising SEQ ID NO: 6; wherein n is 2.

In embodiments, the present disclosure provides a composition comprising an ADC described herein. In embodiments, the composition further comprises at least one excipient, a carrier, and/or a diluent. In embodiments, the composition of the present disclosure is formulated for pharmaceutical use in humans.

In embodiments, the present disclosure provides a method of making an ADC, comprising contacting an anti-EGFR antibody with a synthon according to structural Formula (Ia) D-L-R^{x}, wherein D is a cytotoxic and/or cytostatic agent capable of crossing a cell membrane, L is a linker capable of being cleaved by a lysosomal enzyme, and R^{x} comprises a functional group capable of covalently linking the synthon to the antibody, under conditions in which the synthon covalently links the synthon to the antibody, wherein D is a PBD dimer, and wherein the antibody comprises a heavy chain comprising the amino acid sequence set forth in SEQ ID NO: 1, and a light chain comprising the amino acid sequence set forth in SEQ ID NO: 6.

In embodiments, the present disclosure provides a method of making an ADC, comprising contacting an anti-EGFR antibody with a synthon according to structural Formula (Ia) D-L-R^{x}, wherein D is a cytotoxic and/or cytostatic agent capable of crossing a cell membrane, L is a linker capable of being cleaved by a lysosomal enzyme, and R^{x} comprises a functional group capable of covalently linking the synthon to the antibody, under conditions in which the synthon covalently links the synthon to the antibody, wherein D is a PBD dimer, and wherein the antibody comprises (i) a heavy chain variable region comprising a CDRH1 sequence comprising SEQ ID NO: 3, a CDRH2 sequence comprising SEQ ID NO: 4, and a CDRH3 sequence comprising SEQ ID NO: 5; (ii) a light chain variable region comprising a CDRL1 sequence comprising SEQ ID NO: 8, a CDRL2 sequence comprising SEQ ID NO: 9, and a CDRL3 sequence comprising SEQ ID NO: 10; and (iii) a mutation comprising S239C in a heavy chain constant region, wherein the numbering is in accordance with Kabat. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure provides a method of making an ADC, comprising contacting an anti-EGFR antibody with a synthon according to structural Formula (Ia) D-L-R^{x}, wherein D is a cytotoxic and/or cytostatic agent capable of crossing a cell membrane, L is a linker capable of being cleaved by a lysosomal enzyme, and R^{x} comprises a functional group capable of covalently linking the synthon to the antibody, under conditions in which the synthon covalently links the synthon to the antibody, wherein D is a PBD dimer, and wherein the antibody comprises (i) a heavy chain variable region comprising a CDRH1 sequence comprising SEQ ID NO: 3, a CDRH2 sequence comprising SEQ ID NO: 4, and a CDRH3 sequence comprising SEQ ID NO: 5; (ii) a light chain variable region comprising a CDRL1 sequence comprising SEQ ID NO: 8, a CDRL2 sequence comprising SEQ ID NO: 9, and a CDRL3 sequence comprising SEQ ID NO: 10; and (iii) a mutation comprising S239C in a heavy chain constant region, wherein the numbering is in accordance with Kabat; and wherein R^{x} is a sulfhydryl group or a maleimide-sulfhydryl group. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

In embodiments, the present disclosure provides a method of making an ADC, comprising contacting an anti-EGFR antibody with a synthon according to structural Formula (Ia) D-L-R^{x}, wherein D is a cytotoxic and/or cytostatic agent capable of crossing a cell membrane, L is a linker capable of being cleaved by a lysosomal enzyme, and R^{x} comprises a functional group capable of linking the synthon to the antibody, wherein D is a PBD dimer; wherein L comprises the linker as described in Formula III, IV, V, VI, VII, VIII, or IX; and wherein the antibody comprises (i) a heavy chain variable region comprising a CDRH1 sequence comprising SEQ ID NO: 3, a CDRH2 sequence comprising SEQ ID NO: 4, and a CDRH3 sequence comprising SEQ ID NO: 5; (ii) a light chain variable region comprising a CDRL1 sequence comprising SEQ ID NO: 8, a CDRL2 sequence comprising SEQ ID NO: 9, and a CDRL3 sequence comprising SEQ ID NO: 10; and (iii) a mutation comprising S239C in a heavy chain constant region, wherein the numbering is in accordance with Kabat; and wherein R^{x} is a sulfhydryl group or a maleimide-sulfhydryl group. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR Antibody is a humanized antibody.

In embodiments, the present disclosure provides a method of making an ADC, comprising contacting an anti-EGFR antibody with a synthon according to structural Formula (Ia) D-L-R^{x}, wherein D is a cytotoxic and/or cytostatic agent capable of crossing a cell membrane, L is a linker capable of being cleaved by a lysosomal enzyme, and R^{x} comprises a functional group capable of linking the synthon to the antibody, wherein D is a PBD dimer; wherein L comprises the linker as described in Formula IX; and wherein the antibody comprises (i) a heavy chain variable region comprising a CDRH1 sequence comprising SEQ ID NO: 3, a CDRH2 sequence comprising SEQ ID NO: 4, and a CDRH3 sequence comprising SEQ ID NO: 5; (ii) a light chain variable region comprising a CDRL1 sequence comprising SEQ ID NO: 8, a CDRL2 sequence comprising SEQ ID NO: 9, and a CDRL3 sequence comprising SEQ ID NO: 10; and (iii) a mutation comprising S239C in a heavy chain constant region, wherein the numbering is in accordance with Kabat; and wherein R^{x} is a sulfhydryl group or a maleimide-sulfhydryl group. In embodiments, the anti-EGFR antibody comprises an IgG1 isotype. In embodiments, the heavy chain constant region of the anti-EGFR antibody either lacks a C-terminal lysine or comprises an amino acid other than lysine at a C-terminus of the heavy chain constant region. In embodiments, the anti-EGFR antibody is a humanized antibody.

### BRIEF DESCRIPTION OF THE DRAWINGS

FIG. 1 shows a schematic of EGFR and the regions bound by Ab1 and Ab2 (an antibody having the same six CDR amino acid sequences of cetuximab).

FIG. 2 shows a preparation of AbA (S239C)-PBD. The conjugation process consists of reduction of the interchain disulfides, quantitative oxidation, and conjugation with excess PBD drug linker, as described in Example 2.

FIG. 3 provides the variable heavy (VH) and variable light (VL) chain region amino acid sequences of Ab1 and AbA. CDR sequences within the VH and VL regions are boxed, and differences between the Ab1 VH sequence and the AbA VH sequence are shaded.

FIG. 4 describes the full length light and heavy chains for Ab1 and AbA. Differences between the Ab1 sequence and the AbA sequence in the heavy chain are highlighted.

FIG. 5 shows the flow cytometry analysis of Ab1 and AbA, the S239C mutant forms Ab1(S239C) and AbA(S239C), and the PBD conjugates Ab1(S239C)-PBD and AbA(S239C)-PBD to human cells. Increasing concentrations of antibodies were added to wild-type EGFR-overexpressing (FIG. 5A) and EGFR CA mutant-overexpressing (FIG. 5B) NR6 cells in which the EGFR epitope recognized by Ab1 and AbA is exposed. As shown and described in Example 3, the conjugation of Cys-engineered AbA(S239C) to PBD does not alter the binding properties compared to the parental antibody AbA(S239C) or AbA.

FIG. 6 shows the EGFR number for SW-48 (a colorectal adenocarcinoma cell line that expresses EGFR, >200,000 receptors per cell, IHC H-score 228), NCI-H441 (a lung adenoma xenograft model with moderate to low EGFR expression, ˜100,000 receptors per cell; IHC H-score 150) and LoVo (a KRAS mutant colorectal adenocarcinoma with lower EGFR expression, <100,000 receptors per cell, IHC H-score 140), in comparison to a number of other EGFR-overexpressing cell lines. Cell surface density (antigen binding capacity per cell) was determined by FACS analysis of cell surface antigens on cultured cells using a QIFIT assay with cetuximab.

FIG. 7 shows the improved cytotoxic activity of AbA(S239C)-PBD compared to a corresponding auristatin conjugate (AbA-MMAE) against a panel of tumor cell lines that express different levels of surface EGFR (i.e., low, moderate, or high expression of EGFR). SW-48 (FIG. 7A), NCI-H441 (FIG. 7B), LoVo (FIG. 7C), and A431 (FIG. 7D) tumor cells were plated in 96-well plates with ADCs added at the concentrations shown. After 72 hours at 37° C., cell viability was assessed using an ATPlite Luminescence assay. As shown in FIG. 7A-D, there was improved cytotoxic activity in all four cell lines following treatment with the PBD conjugate AbA(S239C)-PBD as compared to a corresponding auristatin conjugate (AbA-MMAE ADC), for each EGFR expression level.

FIG. 8A is a graph that shows the in vivo efficacy of AbA(S239C)-PBD in the NCI-H441 lung adenocarcinoma xenograft model. Numbers in parentheses represent dose in mg/kg. Arrows represent days of dosing. As shown in FIG. 8A and described in Example 5, AbA(S239C)-PBD, dosed at 0.3 mg/kg, induced complete and durable regressions in 100% of animals.

FIG. 8B is a graph that shows the in vivo efficacy of AbA(S239C)-PBD in the LoVo colorectal adenocarcinoma xenograft tumor model. Numbers in parentheses represent dose in mg/kg. Arrows represent days of dosing. As shown in FIG. 8B and described in Example 5, a corresponding EGFR ADC (AbA-MMAE) showed activity in this model but required dosing at a much higher dose (specifically, a 10 fold higher dose) than AbA(S239C)-PBD.

FIGS. 9A and 9B show the in vivo efficacy of AbA(S239C)-PBD and Ab1(S239C)-PBD in the SW-48 colorectal cancer xenograft tumor model. Numbers in parentheses represent the dose in mg/kg. Arrows represent days of dosing.

FIG. 10A shows the in vivo efficacy of AbA(S239C)-PBD and Ab1(S239C)-PBD in the patient-derived xenograft model CTG-0162 (NSCLC) (FIG. 10A). Numbers in parentheses represent dose in mg/kg, and arrows represent days of dosing. As shown in FIG. 10A and discussed in Example 5, in the CTG-0162 NSCLC model, AbA(S239C)-PBD and Ab1(S239C)-PBD were very effective in inhibiting tumor growth, whereas AbA-MMAE was less efficacious, even though it was dosed ten-fold higher than AbA(S239C)-PBD or Ab1(S239C)-PBD.

FIG. 10B shows the in vivo efficacy of AbA(S239C)-PBD and Ab1(S239C)-PBD in the patient-derived xenograft CTG-0786 head and neck cancer (HNC) model. Numbers in parentheses represent dose in mg/kg, and arrows represent days of dosing. As shown in FIG. 10B and discussed in Example 5, AbA(S239C)-PBD and AbA(S239C)-PBD were effective at inhibiting tumor growth, while the auristatin-based ADC AbA-MMAE required a much higher dose to achieve efficacy.

FIG. 11A is a graph that shows protein aggregation and fragmentation for AbA(S239C). Percent (%) aggregates and % fragments are shown at time “0” (t0) and as percent fragment increase per day and percent aggregate increase per day. As shown and described in Example 6, the in vitro plasma stability of the AbA(S239C) mAb and AbA(239C)-PBD DAR2 was similar to, if not better than, AbA-vcMMAE.

FIG. 11B is a graph that shows protein aggregation and fragmentation for AbA(S239C)-PBD DAR2. Percent (%) aggregates and % fragments are shown at time “0” (t0) and as percent fragment increase per day and percent aggregate increase per day. As shown and described in Example 6, the in vitro plasma stability of the AbA(S239C) mAb and AbA(S239C)-PBD DAR2 was similar to, if not better than, AbA-vcMMAE.

### DETAILED DESCRIPTION

The present disclosure relates to antibody drug conjugates (ADCs) that target EGFR and uses thereof. The ADCs of the present disclosure possess favorable attributes that provide a distinct advantage over other ADCs disclosed in the prior art. For example, the ADCs of the present disclosure are considerably more potent than auristatin-based ADCs using essentially the same antibody backbone, as shown in Examples 3-5 below. That is, the ADCs of the present disclosure (1) show greater potency than corresponding auristatin ADCs when administered at the same dose, and (2) show similar potency to corresponding auristatin ADCs when administered at a considerably lower (i.e., 10 times lower) dose. Moreover, the ADCs of the present disclosure are stable under a variety of conditions, as shown in Example 6 below. The antibodies of the present disclosure also have a low single species drug loading of about 2 (or average drug to antibody ratio of about 2) while retaining a high degree of potency.

Accordingly, the present disclosure pertains to antibody drug conjugates comprising a cytotoxic and/or cytostatic agent (e.g., PBD) linked to an anti-EGFR antibody by way of a linker; compositions comprising the ADCs of the present disclosure; methods of making the ADCs of the present disclosure; and methods of using the ADCs to treat cancer, such as cancers associated with overexpression or amplification of EGFR.

In embodiments, the present disclosure features an ADC comprising the structure of formula (X):

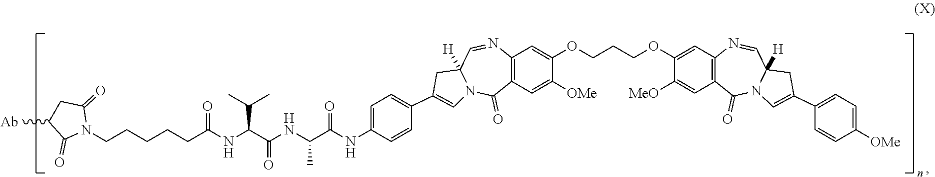

or a salt thereof, wherein Ab comprises an anti-EGFR antibody comprising (i) a heavy chain variable region comprising a heavy chain CDRH1 domain comprising the amino acid sequence set forth in SEQ ID NO: 3; a heavy chain CDRH2 domain comprising the amino acid sequence set forth in SEQ ID NO: 4, and a heavy chain CDRH3 domain comprising the amino acid sequence set forth in SEQ ID NO: 5; (ii) a light chain CDRL1 domain comprising the amino acid sequence set forth in SEQ ID NO: 8; a light chain CDRL2 domain comprising the amino acid sequence set forth in SEQ ID NO: 9; a light chain CDRL3 domain comprising the amino acid sequence set forth in SEQ ID NO: 10, (iii) a mutation comprising S239C in a heavy chain constant region, wherein the numbering is in accordance with Kabat; and (iv) wherein n is 2.

In embodiments, the present disclosure features an ADC comprising the structure of Formula (X):

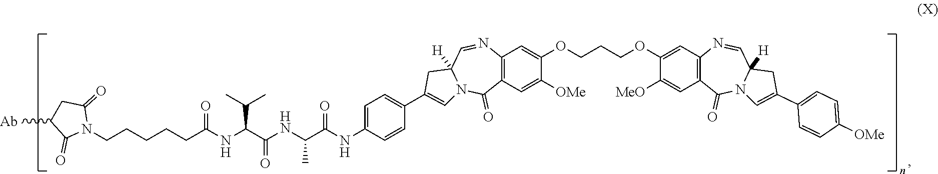

or a salt thereof, wherein Ab comprises an anti-EGFR antibody comprising (i) a heavy chain variable region comprising SEQ ID NO: 2, (ii) a light chain variable region comprising SEQ ID NO: 7; (iii) a mutation comprising S239C in a heavy chain constant region, wherein the numbering is in accordance with Kabat, and (iv) wherein n is 2.

In embodiments, the present disclosure features an ADC comprising the structure of Formula (X):

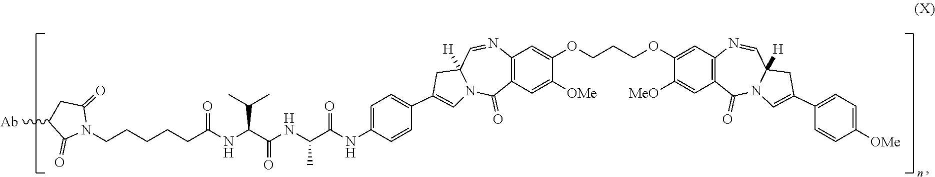

or a salt thereof, wherein Ab comprises an anti-EGFR antibody comprising (i) a heavy chain comprising SEQ ID NO: 1, (ii) a light chain comprising SEQ ID NO: 6; and (iii) wherein n is 2.

In embodiments, the present disclosure features an ADC comprising a cytotoxic and/or cytostatic agent linked to an anti-EGFR antibody by way of a linker, wherein the ADC is a compound according to the structural formula (I):

[D-L-XY]ₙAb (I),

or a salt thereof, wherein D comprises a pyrrolobenzodiazepine (PBD) dimer; L is a linker; Ab is an anti-EGFR antibody comprising a heavy chain comprising the amino acid sequence set forth in SEQ ID NO: 1, and a light chain comprising SEQ ID NO: 6; XY represents a covalent linkage linking linker L to antibody Ab, and n is an integer. In particular, the anti-EGFR ADCs comprising specific linkers and specific cytotoxic and/or cytostatic agents (e.g., a PBD dimer) described herein, exert surprisingly potent anti-tumor activities, in particular when compared to ADCs comprising essentially the same antibody linked to an auristatin. Moreover, the anti-EGFR ADCs of the present disclosure are characterized by a low fixed single species drug loading, which surprisingly results in a highly efficacious ADC in, for example, treating cancer associated with either high or low levels of EGFR expression. As described in the Examples herein, AbA(239C)-PBD is a more potent conjugate than a corresponding AbA-auristatin ADC. As used herein, “AbA” refers to an antibody having a heavy chain comprising SEQ ID NO: 11, and a light chain comprising SEQ ID NO: 6. “AbA (5239C)” refers to an antibody having a heavy chain comprising SEQ ID NO: 1, and a light chain comprising SEQ ID NO: 6. AbA has the same heavy chain sequence as AbA(S239C), but with a serine at position 239 (Kabat numbering).

As will be appreciated by skilled artisans, antibodies and/or binding fragments are “modular” in nature. Throughout the disclosure, various specific embodiments of the various “modules” comprising the antibodies and/or binding fragments are described. As specific non-limiting examples, various specific embodiments of variable heavy chain (VH) CDRs, V_{H} chains, variable light chain (V_{L}) CDRs and V_{L} chains are described. The ADCs disclosed herein are also “modular” in nature. Throughout the disclosure, various specific embodiments of the various “modules” comprising the ADCs are described. As specific non-limiting examples, specific embodiments of antibodies, linkers, and cytotoxic and/or cytostatic agents that may compose the ADCs are described.

The ADCs described herein may be in the form of salts, and in some specific embodiments, pharmaceutically acceptable salts. The ADCs of the disclosure that possess a sufficiently acidic, a sufficiently basic, or both functional groups, can react with any of a number of inorganic bases, and inorganic and organic acids, to form a salt.

Unless otherwise defined herein, scientific and technical terms used in connection with the present disclosure shall have the meanings that are commonly understood by those of ordinary skill in the art.

The terms “anti-Epidermal Growth Factor (EGF) Receptor antibody” or “anti-EGFR antibody”, used interchangeably herein, refer to an antibody that specifically binds to EGFR. An antibody “which binds” an antigen of interest, i.e., EGFR, is one capable of binding that antigen with sufficient affinity such that the antibody is useful in targeting a cell expressing the antigen. In a preferred embodiment, the antibody specifically binds to human EGFR (hEGFR). Examples of anti-EGFR antibodies are disclosed in Example 1 below. Unless otherwise indicated, the term “anti-EGFR antibody” is meant to refer to an antibody which binds to wild type EGFR or any variant of EGFR, such as EGFRvIII.

The amino acid sequence of wild type human EGFR is provided below as SEQ ID NO: 12, where the signal peptide (amino acid residues 1-24) are underlined, and the amino acid residues of the extracellular domain (ECD, amino acid residues 25-645) are highlighted in bold. A truncated wild type ECD of the EGFR (also referred to herein as EGFR(1-525)) is equivalent to amino acids 1-525 of SEQ ID NO: 12. The mature form of wild type EGFR corresponds to the protein without the signal peptide, i.e., amino acid residues 25 to 1210 of SEQ ID NO: 12

| (SEQ ID NO: 12) | |
|---|---|
| 1 | mrpsgtagaa llallaalcp asra leekkv cqgtsnkltq lgtfedhfls |
| | lqrmfnncev |
| 61 | vlgnleityv qrnydlsflk tiqevagyvl ialntverip lenlqiirgn |
| | myyensyala |
| 121 | vlsnydankt glkelpmrnl qeilhgavrf snnpalcnve siqwrdivss |
| | dflsnmsmdf |
| 181 | qnhlgscqkc dpscpngscw gageeneqkl tkiicaqqes grcrgkspsd |
| | cchnqcaagc |
| 241 | tgpresdelv crkfrdeatc kdtcpplmly npttyqmdvn pegkysfgat |
| | cvkkcprnyv |
| 301 | vtdhgscvra cgadsyemee dgvrkckkce gpcrkvcngi gigefkdsls |
| | inatnikhfk |
| 361 | nctsisgdlh ilpvafrgds fthtppldpq eldilktyke itgflliqaw |
| | penrtdlhaf |
| 421 | enleiirgrt kqhgqfslav vslnitsigl rslkeisdgd viisgnknlc |
| | yantinwkkl |
| 481 | fgtsgqktki isnrgensck atgqvchalc spegcwgpep rdcvscrnvs |
| | rgrecvdkcn |
| 541 | llegeprefv enseciqchp eclpqamnit ctgrgpdnci qcahyidgph |
| | cvktcpagvm |
| 601 | genntivwky adaghvchlc hpnctygctg pglegcptng pkipsiatgm |
| | vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd |
| | nphvcrllgi |
| 781 | cltstvglit qlmpfgclld yvrehkdnig sqyllnwcvq iakgmnyled |
| | rrlvhrdlaa |
| 841 | rnvlvktpqh vkitdfglak llgaeekeyh aeggkvpikw malesilhri |
| | ythqsdvwsy |
| 901 | gvtvwelmtf gskpydgipa seissilekg erlpqppict idvymimvkc |
| | wmidadsrpk |
| 961 | freliiefsk mardpqrylv iqgdermhlp sptdsnfyra lmdeedmddv |
| | vdadeylipq |
| 1021 | qgffsspsts rtpllsslsa tsnnstvaci drnglqscpi kedsflqrys sdptgalted |
| 1081 | siddtflpvp eyinqsvpkr pagsvqnpvy hnqpinpaps rdphyqdphs |
| | tavgnpeyin |
| 1141 | tvqptcvnst fdspahwaqk gshqisldnp dyqqdffpke akpngifkgs |
| | taenaeylrv |
| 1201 | apqssefiga |

The amino acid sequence of the ECD of human EGFR is provided below as SEQ ID NO: 13, and includes the signal sequence (underlined).

| (SEQ ID NO: 13) | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss |
| | dflsnmsmdf |
| 181 | qnhlgscqkc dpscpngscw gageencqkl tkiicaqqcs grcrgkspsd |
| | cchnqcaagc |
| 241 | tgpresdclv crkfrdeatc kdtcpplmly npttyqmdvn pegkysfgat |
| | cvkkcprnyv |
| 301 | vtdhgscvra cgadsyemee dgvrkckkce gperkvcngi gigefkdsls |
| | inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs |
| | rgrecvdkch |
| 541 | llegeprefv enseciqchp eclpqamnit ctgrgpdnci qcahyidgph |
| | cvktcpagvm |
| 601 | genntlvwky adaghvchlc hpnctygctg pglegcptng pkips |

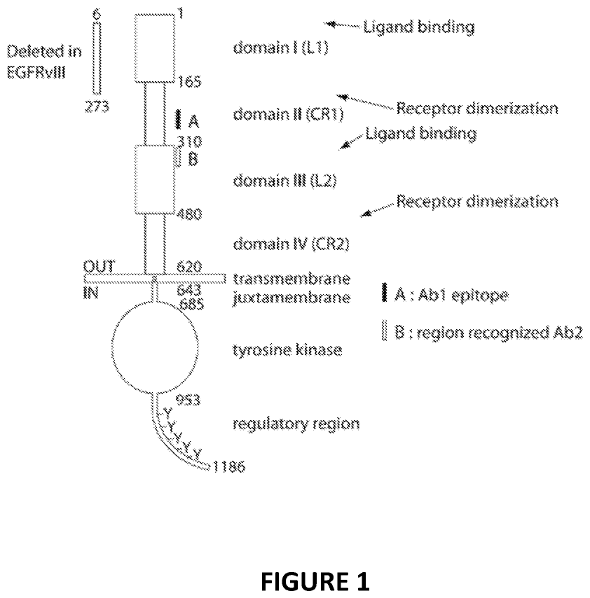

The overall structure of EGFR is described in FIG. 1. The ECD of EGFR has four domains (Cochran et al. (2004) J. Immunol. Methods, 287, 147-158). Domains I and III have been suggested to contribute to the formation of high affinity binding sites for ligands. Domains II and IV are cysteine rich, laminin-like regions that stabilize protein folding and contain a possible EGFR dimerization interface. The figure further shows the regions bound by Ab1 and Ab2. Ab1 is a humanized EGFR antibody having a heavy chain variable region (VH) sequence as provided in SEQ ID NO: 15 (with a CDRH1, CDRH2, and CDRH3 set as set forth in SEQ ID NOS: 16, 17, and 18, respectively) and a light chain variable region (VL) amino acid sequence as provided in SEQ ID NO: 7 (with a CDRL1, CDRL2, and CDRL3 set as set forth in SEQ ID NOS: 8, 9, and 10, respectively). Ab2 is an antibody having the same six CDR amino acid sequences of cetuximab.

EGFR variants may result from gene rearrangement accompanied by EGFR gene amplification. EGFRvIII is the most commonly occurring variant of the EGFR in human cancers (Kuan et al. Endocr Relat Cancer. 8(2):83-96 (2001)). During the process of gene amplification, a 267 amino acid deletion occurs in the extracellular domain of EGFR with a glycine residue inserted at the fusion junction. Thus, EGFRvIII lacks amino acids 6-273 of the extracellular domain of wild type EGFR and includes a glycine residue insertion at the junction. The EGFRvIII variant of EGFR contains a deletion of 267 amino acid residues in the extracellular domain where a glycine is inserted at the deletion junction. The EGFRvIII amino acid sequence is shown below as SEQ ID NO: 14 (the ECD is highlighted in bold and the signal sequence is underlined).

(SEQ ID NO: 14)
mrpsgtagaallallaalcpasra leekkgnyvvtdhgscvracgad
syemeedgvrkckkcegpcrkvcngigigefkdslsinatnikhfknc
tsisgdlhilpvafrgdsfthtppldpqeldilktvkeitgflliqaw
penrtdlhafenleiirgrtkqhgqfslavvslnitslglrslkeisd
gdviisgnknlcyantinwkklfgtsgqktkiisnrgensckatgqvc
halcspegcwgpeprdcvscrnvsrgrecvdkcnllegeprefvense
ciqchpeclpqamnitctgrgpdncigcahyidgphcvktcpagvmge
nntlvwkyadaghvchlchpnctygctgpglegcptngpkipsiatgm
vgalllllvvalgiglfmrrrhivrkrtlrrllqerelvepltpsgea
pnqallrilketefkkikvlgsgafgtvykglwipegekvkipvalke
lreatspkankeildeayvmasvdnphvcrllgicltstvqlitqlmp
fgclldyvrehkdniqsqyllnwcvqiakgmnyledrrlvhrdlaarn
vlvktpqhvkitdfglakllgaeekeyhaeggkvpikwmalesilhri
ythqsdvwsygvtvwelmtfgskpydgipaseissilekgerlpqppi
ctidvymimvkcwmidadsrpkfreliiefskmardpqrylviqgder
mhlpsptdsnfyralmdeedmddvvdadeylipqqgffsspstsrtpl
lsslsatsnnstvacidrnglqscpikedsflqryssdptgaltedsi
ddtflpvpeyinqsvpkrpagsvqnpvyhnqplnpapsrdphyqdphs
tavgnpeylntvqptcvnstfdspahwaqkgshqisldnpdyqqdffp
keakpngifkgstaenaeylrvapqssefiga

EGFRvIII contributes to tumor progression through constitutive signaling in a ligand independent manner. EGFRvIII is not known to be expressed in normal tissues (Wikstrand et al. Cancer Research 55(14): 3140-3148 (1995); Olapade-Olaopa et al. Br J Cancer. 82(1):186-94 (2000)), but shows significant expression in tumor cells, in particular in glioblastoma multiforme (Wikstrand et al. Cancer Research 55(14): 3140-3148 (1995); Ge et al. Int J Cancer. 98(3):357-61 (2002); Wikstrand et al. Cancer Research 55(14): 3140-3148 (1995); Moscatello et al. Cancer Res. 55(23):5536-9 (1995); Garcia de Palazzo et al. Cancer Res. 53(14):3217-20 (1993); Moscatello et al. Cancer Res. 55(23):5536-9 (1995); and Olapade-Olaopa et al. 2(1):186-94 (2000)).

As used herein, the term “antibody” (Ab) refers to an immunoglobulin molecule that specifically binds to, or is immunologically reactive with, a particular antigen, i.e., hEGFR. Antibodies comprise complementarity determining regions (CDRs), also known as hypervariable regions, in both the light chain and heavy chain variable domains. The more highly conserved portions of the variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria, while being deemed to be outside a hypervariable region under a different set of criteria. One or more of these positions can also be found in extended hypervariable regions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies. See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al. unless otherwise indicated.

The term “monoclonal antibody” as used herein is not limited to antibodies produced through hybridoma technology. A monoclonal antibody is derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. In many uses of the present disclosure, including in vivo use of ADCs including anti-EGFR antibodies in humans, chimeric, primatized, humanized, or human antibodies can suitably be used. In embodiments, the anti-EGFR antibodies of the present disclosure are humanized.

“Humanized” forms of non-human (e.g., murine) antibodies are chimeric immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody can also comprise at least a portion of an immunoglobulin constant region (Fc), typically that of a human immunoglobulin consensus sequence. Methods of antibody humanization are known in the art.

Anti-EGFR ADCs of the present disclosure may comprise full length (intact) antibody molecules that are specifically capable of binding EGFR. In embodiments, the ADC of the present disclosure comprises a full length AbA(S239C) antibody.

The term “cytotoxic and/or cytostatic agent”, as used herein, is meant to refer to any agent or drug known to inhibit the growth and/or replication of, and/or kill cells. In one embodiment, the cytotoxic and/or cytostatic agent is a cell-permeating DNA minor groove-binding agent such as a pyrrolobenzodiazepine (“PBD”) and PBD dimers.

The term “antibody drug conjugate” or “ADC” refers to an antibody chemically linked to one or more cytotoxic and/or cytostatic agents. In embodiments, an ADC includes an antibody, cytotoxic and/or cytostatic agent, and a linker that enables attachment or conjugation of the cytotoxic and/or cytostatic agent to the antibody. An ADC of the present disclosure typically has from 1 to 3 cytotoxic and/or cytostatic agents conjugated to the antibody, including a drug loaded species of 1, 2, or 3.

The ADCs disclosed herein may comprise drug molecules and antibody moieties in various stoichiometric molar ratios depending on the configuration of the antibody and, at least in part, on the method used to effect conjugation.

For the purposes of the present disclosure, one skilled in the art would understand that “drug loading” and “drug to antibody ratio” (also referred to as DAR) are distinct. DAR refers to the average molar ratio of drug molecules per antibody in a population of at least two ADC molecules, whereas drug loading refers to the molar ratio of drug molecules per antibody in an individual ADC molecule. Drug loading primarily has relevance for the construction and design of an ADC, whereas DAR primarily has relevance for the therapeutic ADC composition that will be administered to patients.

The term “drug load” or “drug loading” refers to the molar ratio of drug molecules per antibody in an individual ADC molecule. In certain embodiments the drug loading may comprise from 1 to 2, from 1 to 4 drug molecules, from 2-4 drug molecules, from 1-3 drug molecules, or from 2-3 drug molecules (i.e., where for each of the forgoing, the general formula of an ADC molecule is A(-L-D)n, and where n is an integer or a range of integers representing the range of recited drug molecules).

The term “drug to antibody ratio” or “DAR” refers to the weighted average molar ratio of drug molecules per antibody in a population of at least two ADC molecules. Despite the relative conjugate specificity provided by technologies such as engineered antibody constructs, selective cysteine reduction, and post-fabrication purification, a given population of ADCs may comprise ADC molecules having different drug loadings (e.g., ranging from 1 to 8 in the case of an IgG1 antibody). That is, following conjugation, ADC compositions of the invention may comprise a mixture of ADCs with different drug loadings. Such populations may occur for a variety of reasons, but may include batch variability and instances where the chemical conjugation reaction failed to proceed to full completion, among others. Hence, DAR represents the weighted average of drug loadings for the ADC population as a whole (i.e., all the ADC molecules taken together). The ADC population may contain a single predominant or preferred ADC species (e.g., ADCs with a drug loading of 2) with relatively low levels of non-predominant or non-preferred ADC species (e.g., ADCs with a drug loading of 1, 2, 3, or 4, etc.), or it may contain any variety of species having drug loadings of varying proportions (e.g., a DAR of 2.0±0.1, ±0.2, ±0.3, ±0.4, ±0.5, etc.).

In embodiments, the ADCs of the present disclosure comprise an anti-EGFR antibody (e.g., AbA(S239C)) conjugated to a cytotoxic or cytostatic agent (e.g., PBD), having a drug loading of 2. In embodiments, ADC compositions or preparations of the present disclosure comprise an anti-EGFR antibody (e.g., AbA(S239C)) conjugated to a cytotoxic or cytostatic agent (e.g., PBD), wherein the DAR is about 2.

In embodiments, the ADCs of the present disclosure comprise an anti-EGFR antibody comprising a heavy chain variable region comprising a CDR set (CDRH1, CDRH2, CDRH3) as set forth in SEQ ID NOS: 3, 4, and 5, and a light chain variable region comprising a CDR set (CDRL1, CDRL2, CDRL3) as set forth in SEQ ID NOS: 8, 9, and 10. In embodiments, the anti-EGFR antibody is an IgG1 isotype having a heavy chain constant region with a cysteine mutation engineered to provide a conjugation site for PBD. In embodiments, the cysteine mutation is at position 239 of the heavy chain. In embodiments, the mutation is S239C, numbered according to Kabat. The anti-EGFR antibody AbA(S239C) as described herein has a heavy chain variable region comprising CDRH1, CDRH2, and CDRH3 as set forth in SEQ ID NOS: 3, 4, and 5, respectively, and a light chain variable region comprising CDRL1, CDRL2, and CDRL3 as set forth in SEQ ID NOS: 8, 9, and 10, respectively. In embodiments, the anti-EGFR antibody either lacks a C-terminal lysine or comprises an amino acid other than lysine at a C-terminus of the heavy chain constant region.

In embodiments, the ADCs of the present disclosure comprise an anti-EGFR antibody comprising a heavy chain variable region comprising SEQ ID NO: 2, and a light chain variable region comprising SEQ ID NO: 7. In embodiments, the anti-EGFR antibody is an IgG1 isotype having a heavy chain constant region with a cysteine mutation engineered to provide a conjugation site for a PBD. In embodiments, the cysteine mutation is at position 239 of the heavy chain. In embodiments, the cysteine mutation is S239C, numbered according to Kabat. The anti-EGFR antibody AbA(S239C) as described herein has a heavy chain variable region comprising SEQ ID NO: 2, and a light chain variable region comprising SEQ ID NO: 7. In embodiments, the anti-EGFR antibody of the present disclosure either lacks a C-terminal lysine or comprises an amino acid other than lysine at a C-terminus of the heavy chain constant region.

In embodiments, the ADCs of the present disclosure comprise an anti-EGFR antibody comprising a heavy chain comprising SEQ ID NO: 1, and a light chain comprising SEQ ID NO: 6. The anti-EGFR antibody AbA(S239C) as described herein has a heavy chain comprising the amino acid sequence set forth in SEQ ID NO: 1 and a light chain comprising the amino acid sequence set forth in SEQ ID NO: 6. SEQ ID NO: 1 differs from SEQ ID NO: 11 only in that SEQ ID NO: 1 contains the S239C mutation.

Embodiments of the anti-EGFR ADCs described herein may be antibodies or fragments whose sequences have been modified to alter at least one constant region mediated biological effector function. For example, in embodiments, an anti-EGFR ADC may be modified to reduce at least one constant region-mediated biological effector function relative to the unmodified antibody, e.g., reduced binding to the Fc receptor (FcγR). FcγR binding may be reduced by mutating the immunoglobulin constant region segment of the antibody at particular regions necessary for FcγR interactions (See, e.g., Canfield and Morrison, 1991, J. Exp. Med. 173:1483-1491; and Lund et al., 1991, J. Immunol. 147:2657-2662). Reducing FcγR binding may also reduce other effector functions which rely on FcγR interactions, such as opsonization, phagocytosis and antigen-dependent cellular cytotoxicity (“ADCC”).

Antibodies included in anti-EGFR ADCs may have low levels of, or lack, fucose. Antibodies lacking fucose have been correlated with enhanced ADCC activity, especially at low doses of antibody. See Shields et al., 2002, J. Biol. Chem. 277:26733-26740; Shinkawa et al., 2003, J. Biol. Chem. 278:3466-73. Methods of preparing fucose-less antibodies include growth in rat myeloma YB2/0 cells (ATCC CRL 1662). YB2/0 cells express low levels of FUT8 mRNA, which encodes α-1,6-fucosyltransferase, an enzyme necessary for fucosylation of polypeptides.

Antibodies included in anti-EGFR ADCs may include modifications that increase or decrease their binding affinities to the neonatal Fc receptor, FcRn, for example, by mutating the immunoglobulin constant region segment at particular regions involved in FcRn interactions (see, e.g., WO 2005/123780). An anti-EGFR antibody and/or binding fragment may have one or more amino acids inserted into one or more of its hypervariable regions, for example as described in Jung & Plückthun, 1997, Protein Engineering 10:9, 959-966; Yazaki et al., 2004, Protein Eng. Des Sel. 17(5):481-9; and U.S. Pat. App. No. 2007/0280931.

Antibodies may be produced by any of a number of techniques, as described for example in International Publication Nos. WO2015/143382 and WO2010/096434, incorporated by reference in its entirety herein.

Anti-EGFR antibodies and/or binding fragments with high affinity for EGFR, e.g., human EGFR, may be desirable for therapeutic uses. Accordingly, the present disclosure contemplates ADCs comprising anti-EGFR antibodies and/or binding fragments having a high binding affinity to EGFR, and in particular human EGFR. In specific embodiments, the antibodies and/or binding fragments bind EGFR with an affinity of at least about 100 nM, but may exhibit higher affinity, for example, at least about 90 nM, 80 nM, 70 nM, 60 nM, 50 nM, 40 nM, 30 nM, 25 nM, 20 nM, 15 nM, 10 nM, 7 nM, 6 nM, 5 nM, 4 nM, 3 nM, 2 nM, 1 nM, 0.1 nM, 0.01 nM, or even higher. In some embodiments, the antibodies bind EGFR with an affinity in the range of about 1 pM to about 100 nM, or an affinity ranging between any of the foregoing values.

Affinity of antibodies and/or binding fragments for EGFR can be determined using techniques well known in the art or described herein, such as for example, but not by way of limitation, ELISA, isothermal titration calorimetry (ITC), surface plasmon resonance, flow cytometry or fluorescent polarization assays.

Anti-EGFR antibodies can be prepared by recombinant expression of immunoglobulin light and heavy chain genes in a host cell using standard recombinant DNA methodologies known in the art, such as those described in Molecular Cloning; A Laboratory Manual, Second Edition (Sambrook, Fritsch and Maniatis (eds), Cold Spring Harbor, N.Y., 1989). For example, DNAs encoding partial or full-length light and heavy chains are inserted into expression vectors such that the genes are operatively linked to transcriptional and translational control sequences and transformed into a host cell. The antibody light chain gene and the antibody heavy chain gene can be inserted into separate vectors or, more typically, both genes are inserted into the same expression vector, accomplished by methods known in the art. Antibodies can also be produced by chemical synthesis (e.g., by the methods described in Solid Phase Peptide Synthesis, 2nd ed., 1984 The Pierce Chemical Co., Rockford, Ill.).

Anti-EGFR ADCs of the present disclosure generally comprise an anti-EGFR antibody (e.g., AbA (S239C)) having one or more cytotoxic and/or cytostatic agents, which may be the same or different, linked thereto by way of one or more linkers, which may also be the same or different. In embodiments, the anti-EGFR ADCs are compounds according to the structural formula I:

[D-L-XY]ₙ-Ab (I)

or salts thereof, where each “D” represents, independently of the others, a cytotoxic and/or cytostatic agent (“drug”); each “L” represents, independently of the others, a linker; “Ab” represents an anti-EGFR antibody; each “XY” represents a linkage formed between a functional group R^{x} on the linker and a “complementary” functional group R^{y} on the antigen binding moiety; and n represents the number of drugs linked to Ab (i.e., the single species drug loading). Specific embodiments of various anti-EGFR antibodies that may compose ADCs according to structural formula (I) are described above.

In embodiments of the ADCs or salts of structural formula (I), each D is the same and/or each L is the same.

Specific embodiments of cytotoxic and/or cytostatic agents (D) and linkers (L) that may compose the anti-EGFR ADCs, are described in more detail below.

In embodiments, the ADC has the structure of formula (I), or a salt thereof, wherein D comprises a pyrrolobenzodiazapine (PBD) dimer; L is a linker; Ab is an antibody comprising SEQ ID NO: 1; XY represents a covalent linkage linking linker L to antibody Ab; and n is any integer. In embodiments, n is 2 or 4. In embodiments, n is 2.

In embodiments, where the DAR of the ADC refers to the average molar ratio of drug molecules per antibody in a population of at least two ADC molecules, the DAR is about 2. In this context, the term “about” means an amount within ±7.5% of the actual value. That is, “about 2” means 1.85, 1.86, 1.87, 1.88, 1.89, 1.90, 1.91, 1.92, 1.93, 1.94, 1.95, 1.96, 1.97, 1.98, 1.99, 2.00, 2.01, 2.02, 2.03, 2.04, 2.05, 2.06, 2.07, 2.08, 2.09, 2.10, 2.11, 2.12, 2.13, 2.14, 2.15, and any intervening ranges.

Additional details regarding drugs (D of Formula I) and linkers (L of Formula I) that may be used in the ADCs of the present disclosure, as well as alternative ADCs structures, are described below. In embodiments, the cytotoxic and/or cytostatic agent is a pyrrolobenzodiazepine (PBD), e.g., a PBD dimer.

The structures of PBDs can be found, for example, in U.S. Patent Application Pub. Nos. 2013/0028917 and 2013/0028919, and in WO 2011/130598 A1, each of which are incorporated herein by reference in their entirety. The generic structure of a PBD is provided below as Formula (II).

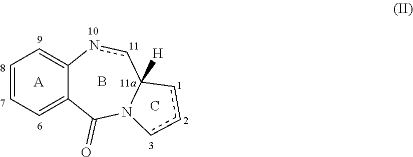

PBDs differ in the number, type and position of substituents, in both their aromatic A rings and pyrrolo C rings, and in the degree of saturation of the C ring. In the B-ring, there is generally an imine (N═C), a carbinolamine (NH—CH(OH)), or a carbinolamine methyl ether (NH—CH(OMe)) at the N10-C11 position which is the electrophilic center responsible for alkylating DNA. All of the known natural products have an (S)-configuration at the chiral C11a position that provides a right-handed twist when viewed from the C ring towards the A ring. The PBD examples provided herein may be conjugated to the anti-EGFR antibodies of the present disclosure. Further examples of PBDs that may be conjugated to the anti-EGFR antibodies of the present disclosure can be found, for example, in U.S. Patent Application Publication Nos. 2013/0028917 A1 and 2013/0028919 A1, in U.S. Pat. No. 7,741,319 B2, and in WO 2011/130598 A1 and WO 2006/111759 A1, each of which are incorporated herein by reference in their entirety.

In the anti-EGFR ADCs described herein, the cytotoxic and/or cytostatic agents are linked to the antibody by way of linkers. The linkers may be short, long, hydrophobic, hydrophilic, flexible, or rigid, and may be composed of segments that independently have one or more of the above-mentioned properties such that the linker may include segments having different properties. The linkers may be polyvalent such that they covalently link more than one agent to a single site on the antibody, or monovalent such that covalently they link a single agent to a single site on the antibody.

In certain embodiments, the linker selected is cleavable in vivo. Cleavable linkers may include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is noncleavable. In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate drug release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone, remain intact during systemic circulation in the blood's neutral pH environment (pH 7.3-7.5) and undergo hydrolysis and release the drug once the ADC is internalized into mildly acidic endosomal (pH 5.0-6.5) and lysosomal (pH 4.5-5.0) compartments of the cell. This pH dependent release mechanism has been associated with nonspecific release of the drug. To increase the stability of the hydrazone group of the linker, the linker may be varied by chemical modification, e.g., substitution, allowing tuning to achieve more efficient release in the lysosome with a minimized loss in circulation.

Hydrazone-containing linkers may contain additional cleavage sites, such as additional acid-labile cleavage sites and/or enzymatically labile cleavage sites. ADCs including exemplary hydrazone-containing linkers include the following structures of Formulas (III), (IV), and (V):

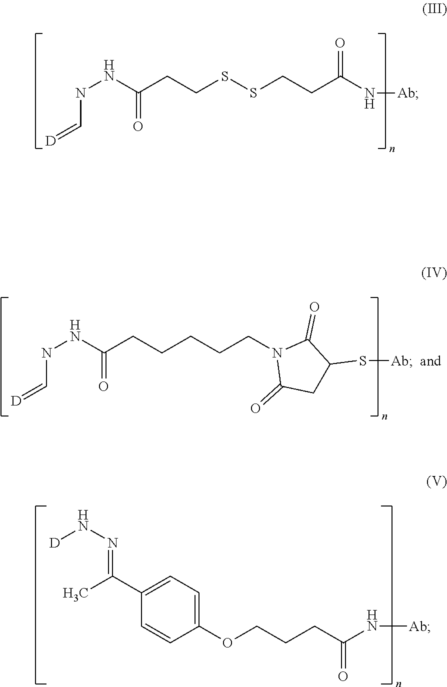

or a salt thereof, wherein D and Ab represent the cytotoxic and/or cytostatic agent (drug) and antibody, respectively, and n represents the number of drug-linkers linked to the antibody. In certain linkers such as that of (Formula (III)), the linker comprises two cleavable groups—a disulfide and a hydrazone moiety. For such linkers, effective release of the unmodified free drug requires acidic pH or disulfide reduction and acidic pH. Linkers such as those of Formula (IV) and (V) have been shown to be effective with a single hydrazone cleavage site.

Other acid-labile groups that may be included in linkers include cis-aconityl-containing linkers. cis-Aconityl chemistry uses a carboxylic acid juxtaposed to an amide bond to accelerate amide hydrolysis under acidic conditions.

Cleavable linkers may also include a disulfide group. Disulfides are thermodynamically stable at physiological pH and are designed to release the drug upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonably stable in circulation, selectively releasing the drug in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hinderance adjacent to the disulfide bond.

ADCs including exemplary disulfide-containing linkers include the following structures of Formulas (VI), (VII), and (VIII):

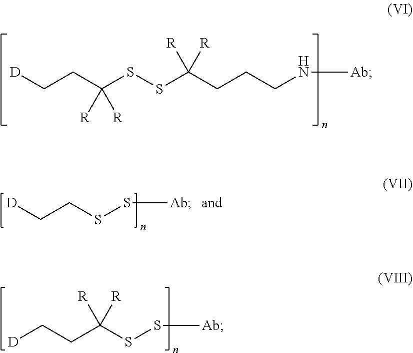

or a salt thereof, wherein D and Ab represent the drug and antibody, respectively, n represents the number of drug-linkers linked to the antibody, and R is independently selected at each occurrence from hydrogen or alkyl, for example. In certain embodiments, increasing steric hinderance adjacent to the disulfide bond increases the stability of the linker. Structures such as (VI) and (VIII) show increased in vivo stability when one or more R groups is selected from a lower alkyl such as methyl.

Another type of cleavable linker that may be used is a linker that is specifically cleaved by an enzyme. Such linkers are typically peptide-based or include peptidic regions that act as substrates for enzymes. Peptide based linkers tend to be more stable in plasma and extracellular milieu than chemically labile linkers. Peptide bonds generally have good serum stability, as lysosomal proteolytic enzymes have very low activity in blood due to endogenous inhibitors and the unfavorably high pH value of blood compared to lysosomes. Release of a drug from an antibody occurs specifically due to the action of lysosomal proteases, e.g., cathepsin and plasmin. These proteases may be present at elevated levels in certain tumor cells.

In exemplary embodiments, the cleavable peptide is selected from tetrapeptides such as Gly-Phe-Leu-Gly, Ala-Leu-Ala-Leu, or dipeptides such as Val-Cit, Val-Ala, Met-(D)Lys, Asn-(D)Lys, Val-(D)Asp, Phe-Lys, Ile-Val, Asp-Val, His-Val, NorVal-(D)Asp, Ala-(D)Asp, Met-Lys, Asn-Lys, Ile-Pro, Me3Lys-Pro, PhenylGly-(D)Lys, Met-(D)Lys, Asn-(D)Lys, Pro-(D)Lys, Met-(D)Lys, Asn-(D)Lys, Met-(D)Lys, Asn-(D)Lys. In embodiments, the cleavable peptide is Val-Ala. In embodiments, the linker is a maleimidocaproyl-valine-alanine (mc-Val-Ala) linker. In certain embodiments, dipeptides are preferred over longer polypeptides due to hydrophobicity of the longer peptides.

A variety of dipeptide-based cleavable linkers useful for linking drugs such as doxorubicin, mitomycin, campotothecin, tallysomycin and auristatin/auristatin family members to antibodies have been described (see, Dubowchik et al., 1998, J. Org. Chem. 67:1866-1872; Dubowchik et al., 1998, Bioorg. Med. Chem. Lett. 8(21):3341-3346; Walker et al., 2002, Bioorg. Med. Chem. Lett. 12:217-219; Walker et al., 2004, Bioorg. Med. Chem. Lett. 14:4323-4327; and Francisco et al., 2003, Blood 102:1458-1465, Dornina et al., 2008, Bioconjugate Chemistry 19:1960-1963, of each of which is incorporated herein by reference). All of these dipeptide linkers, or modified versions of these dipeptide linkers, may be used in the ADCs described herein. Other dipeptide linkers that may be used include those found in ADCs such as Seattle Genetics' Brentuximab Vendotin SGN-35 (Adcetris™), Seattle Genetics SGN-75 (anti-CD-70, Val-Cit-MMAF), Celldex Therapeutics glembatumumab (CDX-011) (anti-NMB, Val-Cit-MMAE), and Cytogen PSMA-ADC (PSMA-ADC-1301) (anti-PSMA, Val-Cit-MMAE).

Enzymatically cleavable linkers may include a self-immolative spacer to spatially separate the drug from the site of enzymatic cleavage. The direct attachment of a drug to a peptide linker can result in proteolytic release of an amino acid adduct of the drug, thereby impairing its activity. The use of a self-immolative spacer allows for the elimination of the fully active, chemically unmodified drug upon amide bond hydrolysis.

One self-immolative spacer is the bifunctional para-aminobenzyl alcohol group, which is linked to the peptide through the amino group, forming an amide bond, while amine containing drugs may be attached through carbamate functionalities to the benzylic hydroxyl group of the linker (PABC). The resulting prodrugs are activated upon protease-mediated cleavage, leading to a 1,6-elimination reaction releasing the unmodified drug, carbon dioxide, and remnants of the linker group. The following scheme depicts the fragmentation of p-amidobenzyl ether and release of the drug:

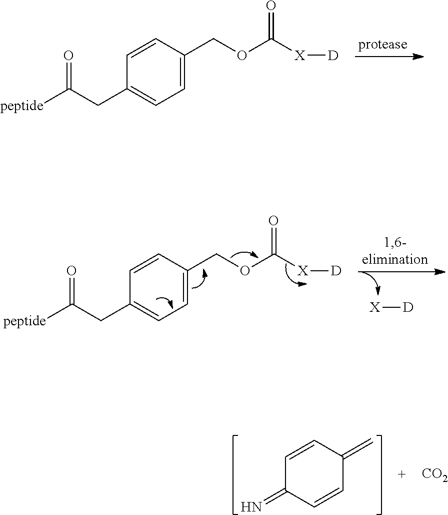

wherein X-D represents the unmodified drug.

Heterocyclic variants of this self-immolative group have also been described. See for example, U.S. Pat. No. 7,989,434, incorporated herein by reference.

In some embodiments, the enzymatically cleavable linker is a β-glucuronic acid-based linker. Facile release of the drug may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an ADC to undergo aggregation due to the hydrophilic nature of β-glucuronides. In some embodiments, β-glucuronic acid-based linkers are preferred as linkers for ADCs linked to hydrophobic drugs. The following scheme depicts the release of the drug from and ADC containing a β-glucuronic acid-based linker:

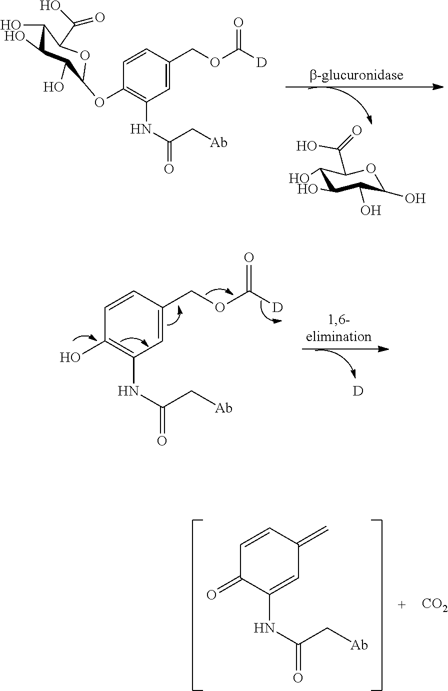

A variety of cleavable β-glucuronic acid-based linkers useful for linking drugs such as auristatins, camptothecin and doxorubicin analogues, CBI minor-groove binders, and psymberin to antibodies have been described (see, see Nolting, Chapter 5 “Linker Technology in Antibody-Drug Conjugates,” In: Antibody Drug Conjugates: Methods in Molecular Biology, vol. 1045, pp. 71-100, Laurent Ducry (Ed.), Springer Science & Business Medica, LLC, 2013; Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255, each of which is incorporated herein by reference). All of these β-glucuronic acid-based linkers may be used in the anti-EGFR ADCs described herein.

In a one embodiment, the linker used in the ADCs of the present disclosure is shown below as Formula (IX), wherein Y is Val, Z is Ala, D is the drug (e.g., a PBD dimer), and q is 1, 2, 3, 4, 5, 6, 7, or 8:

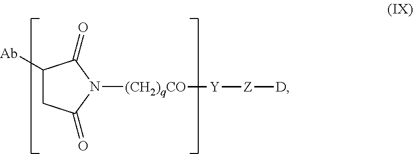

or a salt thereof. In embodiments, q is 5.

In one aspect, the present disclosure describes an ADC comprising a cytotoxic and/or cytostatic agent linked to an antibody by way of a linker, wherein the antibody drug conjugate is a compound according to the structural Formula (I), or a salt thereof, wherein D comprises a pyrrolobenzodiazepine (PBD) dimer; L is a linker; Ab is an antibody comprising SEQ ID NO: 1; XY represents a covalent linkage linking linker L to antibody Ab; and n is any integer. In one embodiment, XY represents a covalent linkage linking linker L to antibody Ab, where the XY is a linkage formed with a sulfhydryl group on antibody Ab. In another embodiment, XY is a maleimide-sulfhydryl linkage.

In certain embodiments, the ADC of the present disclosure comprises the structure of Formula (X):

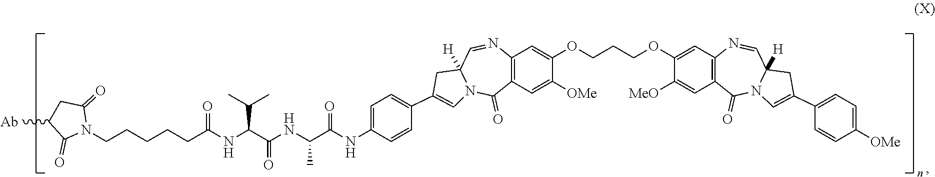

or a salt thereof, wherein Ab is an antibody comprising a heavy chain variable region comprising a CDR set (CDRH1, CDRH2, and CDRH3) as set forth in SEQ ID NOS: 3, 4, and 5, and a light chain variable region comprising a CDR set (CDRL1, CDRL2, and CDRL3) as set forth in SEQ ID NOS: 8, 9, and 10, and n is 2 or 4. In embodiments, the anti-EGFR antibody is an IgG₁ isotype having a constant region with cysteine mutation engineered to provide a conjugation site for a PBD. In one embodiment, the cysteine mutation is at position 239 of the heavy chain. In embodiments, the mutation is S239C, wherein the numbering is in accordance with Kabat. In one embodiment, n is about 2 or about 4. In embodiments, n is about 2. In embodiments, the heavy chain constant region of the anti-EGFR antibody either lacks a C-terminal lysine or comprises an amino acid other than lysine at a C-terminus of the heavy chain constant region.

In embodiments, the ADC of the present disclosure comprises the structure of formula (X),

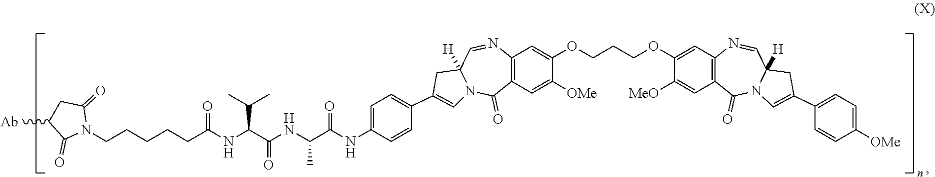

or a salt thereof, wherein Ab is an antibody comprising a heavy chain variable region comprising the amino acid sequence set forth in SEQ ID NO: 2, and a light chain variable region comprising the amino acid sequence set forth in SEQ ID NO: 7. In embodiments, the anti-EGFR antibody is an IgG₁ isotype having a constant region with cysteine mutation engineered to provide a conjugation site for a PBD. In embodiments, the cysteine mutation is at position 239 of the heavy chain. In embodiments, the cysteine mutation is S239C, wherein the numbering is in accordance with Kabat. In one embodiment, n is about 2 or about 4. In another embodiment, n is about 2. In embodiments, the heavy chain constant region of the anti-EGFR antibody either lacks a C-terminal lysine or comprises an amino acid other than lysine at a C-terminus of the heavy chain constant region.

In embodiments, the ADC of the present disclosure comprises the structure of Formula (X):

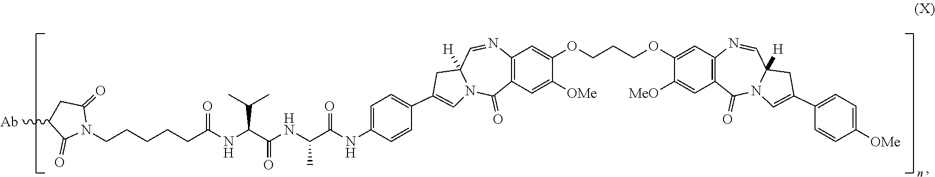

or a salt thereof, wherein Ab is an antibody comprising a heavy chain comprising the amino acid sequence set forth in SEQ ID NO: 1 and a light chain comprising the amino acid sequence set forth in SEQ ID NO: 6. In embodiments, n is about 2 to about 4. In embodiments, n is about 2 or about 4. In embodiments, n is about 2.

The ADCs of the present disclosure may be synthesized using chemistries that are known in the art. The chemistries selected will depend upon, among other things, the identity of the cytotoxic and/or cytostatic agent(s), the linker and the groups used to attach linker to the antibody. Generally, ADCs according to Formula (I) may be prepared according to the following scheme:

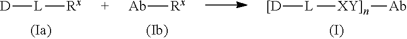

where D, L, Ab, XY and n are as previously defined above, and R^{x} and R^{y} represent complementary groups capable of forming covalent linkages with one another, as discussed above.

The identities of groups R^{x} and R^{y} will depend upon the chemistry used to link synthon D-L-R^{x} to the antibody. Generally, the chemistry used should not alter the integrity of the antibody, for example its ability to bind its target. Preferably, the binding properties of the conjugated antibody will closely resemble those of the unconjugated antibody. A variety of chemistries and techniques for conjugating molecules to biological molecules such as antibodies are known in the art and in particular to antibodies, are well-known. See, e.g., Amon et al., “Monoclonal Antibodies For Immunotargeting Of Drugs In Cancer Therapy,” in: Monoclonal Antibodies And Cancer Therapy, Reisfeld et al. Eds., Alan R. Liss, Inc., 1985; Hellstrom et al., “Antibodies For Drug Delivery,” in: Controlled Drug Delivery, Robinson et al. Eds., Marcel Dekker, Inc., 2nd Ed. 1987; Thorpe, “Antibody Carriers Of Cytotoxic Agents In Cancer Therapy: A Review,” in: Monoclonal Antibodies '84: Biological And Clinical Applications, Pinchera et al., Eds., 1985; “Analysis, Results, and Future Prospective of the Therapeutic Use of Radiolabeled Antibody In Cancer Therapy,” in: Monoclonal Antibodies For Cancer Detection And Therapy, Baldwin et al., Eds., Academic Press, 1985; Thorpe et al., 1982, Immunol. Rev. 62:119-58; PCT publication WO 89/12624. Any of these chemistries may be used to link the synthons to an antibody.

A number of functional groups R^{x} and chemistries useful for linking synthons to accessible lysine residues are known, and include by way of example and not limitation NHS-esters and isothiocyanates.

A number of functional groups R^{x} and chemistries useful for linking synthons to accessible free sulfhydryl groups of cysteine residues are known, and include by way of example and not limitation haloacetyls and maleimides.

However, conjugation chemistries are not limited to available side chain groups. Side chains such as amines may be converted to other useful groups, such as hydroxyls, by linking an appropriate small molecule to the amine. This strategy can be used to increase the number of available linking sites on the antibody by conjugating multifunctional small molecules to side chains of accessible amino acid residues of the antibody. Functional groups R^{x} suitable for covalently linking the synthons to these “converted” functional groups are then included in the synthons.

An antibody may also be engineered to include amino acid residues for conjugation. An approach for engineering antibodies to include non-genetically encoded amino acid residues useful for conjugating drugs in the context of ADCs is described by Axup et al., 2012, Proc Natl Acad Sci USA. 109(40):16101-16106, as are chemistries and functional groups useful for linking synthons to the non-encoded amino acids.

Typically, the synthons are linked to the side chains of amino acid residues of the antibody, including, for example, the primary amino group of accessible lysine residues or the sulfhydryl group of accessible cysteine residues. Free sulfhydryl groups may be obtained by reducing interchain disulfide bonds.

For linkages where R^{y} is a sulfhydryl group (for example, when R^{x} is a maleimide), the antibody is generally first fully or partially reduced to disrupt interchain disulfide bridges between cysteine residues. Specific cysteine residues and interchain disulfide bridges, if present in the antibody heavy chain, may be reduced for attachment of drug-linker synthons including a group suitable for conjugation to a sulfhydryl group, and include by way of example and not limitation: residues C233, C239, and C242 (Kabat numbering system; corresponding to residues C220, C226, and C229 Eu numbering) on the human IgG₁ heavy chain, and residue C214 (Kabat numbering system) on the human Ig kappa light chain. In instances where an antibody heavy chain does not contain a cysteine residue at an attachment site, however, the antibody can be engineered to contain a cysteine at a given position, e.g., position 239.

Cysteine residues for synthon attachment that do not participate in disulfide bridges may be engineered into an antibody by mutation of one or more codons. Reducing these unpaired cysteines yields a sulfhydryl group suitable for conjugation. Preferred positions for incorporating engineered cysteines include, by way of example and not limitation, positions S112C, S113C, A114C, S115C, A176C, 5180C, S239C, S252C, V286C, V292C, S357C, A359C, S398C, S428C (Kabat numbering) on the human IgG₁ heavy chain and positions V110C, S114C, S121C, S127C, S168C, V205C (Kabat numbering) on the human Ig kappa light chain (see, e.g., U.S. Pat. Nos. 7,521,541, 7,855,275 and 8,455,622). In one embodiment, residue 5239 (Kabat numbering system) is mutated to a cysteine to allow conjugation of a PBD to antibody AbA. This mutation is referred to herein as “S239C”.

In certain embodiments, the ADCs of the present disclosure have a drug loading of 2, via the engineered cysteines.

In certain embodiments, the instant disclosure features a method of making an ADC, comprising contacting an antibody heavy and light chains set forth in SEQ ID NOs:1 and 6, respectively, with a synthon according to structural formula (Ia), where D is a cytotoxic and/or cytostatic agent capable of crossing a cell membrane, L is a linker capable of being cleaved by a lysosomal enzyme, and R^{x} comprises a functional group capable of covalently linking the synthon to the antibody, under conditions in which the synthon covalently links the synthon to the antibody, wherein D is, e.g., a PBD dimer.

As will be appreciated by skilled artisans, the number of cytotoxic and/or cytostatic agents linked to an antibody molecule may vary, such that an ADC preparation may be heterogeneous in nature, where some antibodies in the preparation contain one linked agent, some two, some three, etc. (and some none). The degree of heterogeneity will depend upon, among other things, the chemistries used for linking the cytotoxic and/or cytostatic agents. For example, where the antibodies are reduced to yield sulfhydryl groups for attachment, heterogenous mixtures of antibodies having zero, 2, 4, 6 or 8 linked agents per molecule are often produced. Furthermore, by limiting the molar ratio of attachment compound, antibodies having zero, 1, 2, 3, 4, 5, 6, 7 or 8 linked agents per molecule are often produced. Thus, it will be understood that depending upon context, stated drug antibody ratios (DARs) may be averages for a collection of antibodies. For example, “DAR4” refers to an ADC preparation that has not been subjected to purification to isolate specific DAR peaks and comprises a heterogeneous mixture of ADC molecules having different numbers of cytostatic and/or cytotoxic agents attached per antibody (e.g., single species drug loading of 0, 2, 4, 6, 8 agents per antibody), but has an average drug-to-antibody ratio of 4.

Heterogeneous ADC preparations may be processed, for example, by hydrophobic interaction chromatography (“HIC”) to yield preparations enriched in an ADC having a specified DAR of interest (or a mixture of two or more specified DARs). Such enriched preparations are designed herein as “EX,” where “E” indicates the ADC preparation has been processed and is enriched in an ADC population having a specific DAR and “X” represents the number of cytostatic and/or cytotoxic agents linked per ADC molecule. Preparations enriched in a mixture of ADCs having two specific DARs are designated “EX/EY,” three specific DARs “EX/EY/EZ” etc., where “E” indicates the ADC preparation has been processed to enrich the specified species and “X,” “Y” and “Z” represent the species enriched. As specific examples, “E2” refers to an ADC preparation that has been enriched to contain primarily ADCs having two cytostatic and/or cytotoxic agents linked per ADC molecule. “E4” refers to an ADC preparation that has been enriched to contain primarily ADCs having four cytostatic and/or cytotoxic agents linked per ADC molecule. “E2/E4” refers to an ADC preparation that has been enriched to contain primarily two ADC populations, one having two cytostatic and/or cytotoxic agents linked per ADC molecule and another having four cytostatic and/or cytotoxic agents linked per ADC molecule.

As used herein, enriched “E” preparations will generally be at least about 80% pure in the stated DAR ADCs, although higher levels of purity, such as purities of at least about 85%, 90%, 95%, 98%, or even higher, may be obtainable and desirable. For example, an “EX” preparation will generally be at least about 80% pure in ADCs having X cytostatic and/or cytotoxic agents linked per ADC molecule. For “higher order” enriched preparations, such as, for example, “EX/EY” preparations, the sum total of ADCs having X and Y cytostatic and/or cytotoxic agents linked per ADC molecule will generally comprise at least about 80% of the total ADCs in the preparation. Similarly, in an enriched “EX/EY/EZ” preparation, the sum total of ADCs having X, Y and Z cytostatic and/or cytotoxic agents linked per ADC molecule will comprise at least about 80% of the total ADCs in the preparation.

Purity may be assessed by a variety of methods, as is known in the art. As a specific example, an ADC preparation may be analyzed via HPLC or other chromatography and the purity assessed by analyzing areas under the curves of the resultant peaks.

In embodiments, the present disclosure comprises a heterogenous composition comprising AbA(S239C)-PBD ADCs having a DAR of 2 (DAR E2), wherein the DAR E2 species is present at >80 percent (>80, 81, 82, 83, 84, 85, 86, 87, 88, 89, 90, 91, 92, 93, 94, 95, 96, 97, 98, 99 percent) of all ADCs in the composition. For example, in embodiments, the application comprises a heterogeneous composition comprising AbA(S239C)-PBD ADCs having a DAR of 2 (DAR E2), wherein the DAR E2 species is present at >85 percent (85, 86, 87, 88, 89, 90, 91, 92, 93, 94, 95, 96, 97, 98, 99 percent) of the population of all ADCs in the composition. In embodiments, the application comprises a heterogeneous composition comprising AbA(S239C)-PBD ADCs having a DAR of 2 (DAR E2), wherein the DAR E2 species is present at >90 percent (90, 91, 92, 93, 94, 95, 96, 97, 98, 99 percent) of the population of all ADCs in the composition.

In certain embodiments, the DAR of the ADC of the present disclosure is about 2 or about 4. In further embodiments, the DAR of the ADC of the present disclosure is about 2.

The ADCs described herein may be in the form of pharmaceutical compositions comprising the ADC and one or more carriers, excipients, and/or diluents. The compositions may be formulated for specific uses, such as for veterinary uses or pharmaceutical uses in humans.

### BRIEF DESCRIPTION OF THE SEQUENCES

Incorporated by reference herein in its entirety is a Sequence Listing entitled Sequence Listing 12389, comprising SEQ ID NO: 1 through SEQ ID NO: 20, which includes the nucleic acid and/or amino acid sequences disclosed herein. The sequence listing has been submitted herewith in ASCII text format. The sequence was first created on Aug. 31, 2018, and is 45.1 KB in size.

### EXAMPLES

The following Examples, which highlight certain features and properties of exemplary embodiments of anti-EGFR ADCs are provided for purposes of illustration, and not limitation.

It should be noted that, unless otherwise described, the approximate DAR of the PBD ADCs described in the examples is about 2.

### Example 1. Generation of Anti-EGFR AbA (S239C)

Antibody 1 (Ab1) is a humanized anti-EGFR antibody. The heavy chain amino acid sequence of Ab1 is described in SEQ ID NO: 20. The heavy chain variable region is italicized (SEQ ID NO: 15) and the CDRs are underlined.

SEQ ID NO: 20
QVQLQESGPGLVKPSQTLSLTCTVSGYSISSDFAWNWIRQPPGKGLEW
MGYISYSGNTRYQPSLKSRITISRDTSKNQFFLKLNSVTAADTATYYC
VTAGRGFPYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK

The heavy chain variable region (VH) amino acid sequence of Ab1 is provided below as SEQ ID NO: 15. The VH CDR amino acid sequences of Ab1 are underlined below, and are as follows: GYSISSDFAWN (VH CDR1; SEQ ID NO: 16); YISYSGNTRYQPSLKS (VH CDR2; SEQ ID NO: 17); and AGRGFPY (VH CDR3; SEQ ID NO: 18).

Ab1 VH Sequence
(SEQ ID NO: 15)
QVQLQESGPGLVKPSQTLSLTCTVSGYSISSDFAWNWIRQPPGKGLEW
MGYISYSGNTRYQPSLKSRITISRDTSKNQFFLKLNSVTAADTATYYC
VTAGRGFPYWGQGTLVTVSS

The light chain variable region (VL) amino acid sequence of Ab1 is provided below as SEQ ID NO: 7. The VL CDR amino acid sequences of Ab1 are underlined below and are as follows: HSSQDINSNIG (VL CDR1; SEQ ID NO: 8); HGTNLDD (VL CDR2; SEQ ID NO: 9); and VQYAQFPWT (VL CDR3; SEQ ID NO: 10).

Ab1 VL Sequence
(SEQ ID NO: 7)
DIQMTQSPSSMSVSVGDRVTITCHSSQDINSNIGWLQQKPGKSFKGLI
YHGTNLDDGVPSRFSGSGSGTDYTLTISSLQPEDFATYYCVQYAQFPW
TFGGGTKLEIK

A screen was performed to identify anti-EGFR antibodies having improved properties over Ab1. The details of the identification of Ab1 variants are described in, for example, WO2015/143382, which is incorporated by reference herein in its entirety.

One of the identified Ab1 variant antibodies was AbA. AbA has the same variable light chain sequence as Ab1 (SEQ ID NO: 7), including the same CDR1, CDR2, and CDR3 amino acid sequences (described in SEQ ID NOs: 8, 9, and 10, respectively). The VH amino acid sequence of AbA is provided below in SEQ ID NO: 2. The VH CDR amino acid sequences of AbA are as follows: GYSISRDFAWN (CDR1; SEQ ID NO: 3); YISYNGNTRYQPSLKS (CDR2; SEQ ID NO: 4); and ASRGFPY (CDR3; SEQ ID NO: 5), and are underlined below. Residues that are different in the heavy chain variable region of AbA versus Ab1 are shown below in bold.

AbA VH Amino Acid Sequence
(SEQ ID NO: 2)
EVQLQESGPGLVKPSQTLSLTCTVSGYSISRDFAWNWIRQPPGKGLEW
MGYISYNGNTRYQPSLKSRITISRDTSKNQFFLKLNSVTAADTATYYC
VTASRGFPYWGQGILVTVSS

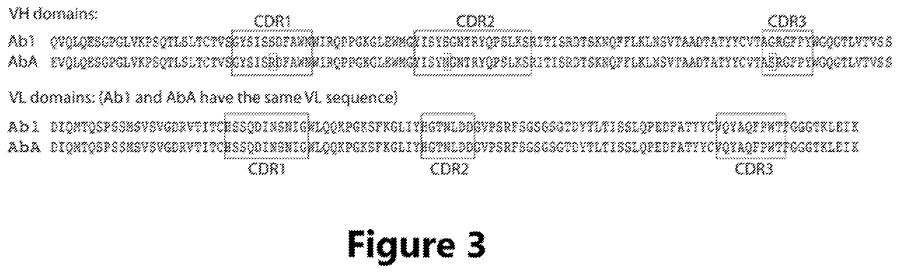

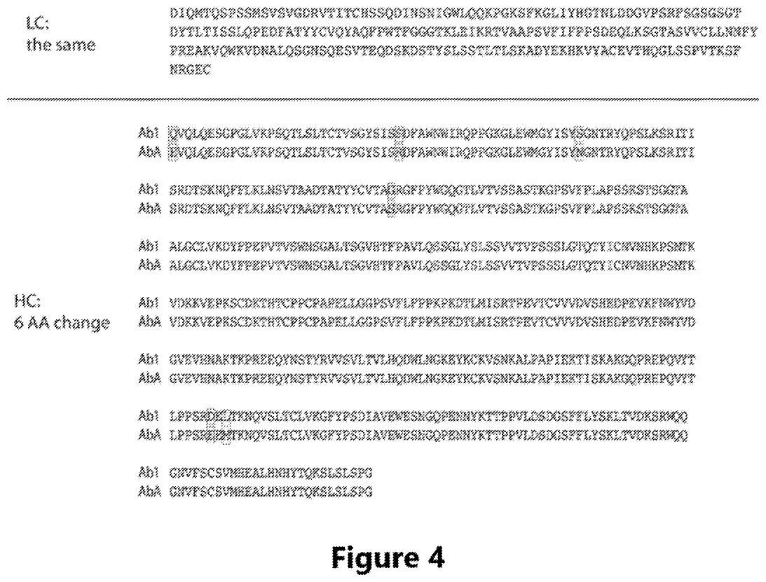

FIG. 3 and FIG. 4 provide an alignment of the amino acid sequences of the VH and VL regions (FIG. 3) and the complete heavy and light chains (FIG. 4) of Ab1 and AbA. The light chain amino acid sequences of Ab1 and AbA are the same (SEQ ID NO: 6). The heavy chain amino acid sequences of Ab1 and AbA, however, have six amino acid differences between the two sequences, three of which are in the CDRs. Differences between the Ab1 VH amino acid sequence and the AbA VH amino acid sequence are shaded in FIG. 3 and are found in each of the VH CDRs. The CDR1 domain of the variable heavy chain of AbA included an amino acid change from a serine (Ab1) to an arginine. The CDR2 domain of the variable heavy chain included an amino acid change from a serine in Ab1 to an asparagine in AbA. Finally, the CDR3 domain of the variable heavy chain included an amino acid change from a glycine in Ab1 to a serine in AbA. Two of the amino acid changes within AbA are in the constant region of the heavy chain (D354E and L356M). The Fc region amino acid mutations in AbA represent human IgG allotype changes from a z, a allotype to a z, non-a allotype. In addition to the other changes, the first amino acid was changed from a glutamine (Q) to a glutamic acid (E), as described, for example, in FIG. 3. Thus, the AbA antibody contains three amino acid differences in the complementarity determining region relative to the Ab1 antibody. However, AbA has improved binding affinity over Ab1 for EGFR, and also exhibits unique in vitro and in vivo characteristics relative to Ab1, as described in, for example, International Application No. WO2015/143382.

Following identification of anti-EGFR antibody AbA, the antibody was modified in order to engineer site-specific conjugation sites of the warhead PBD. Specifically, an engineered cysteine antibody (C239) was generated using common techniques of one of skill in the art in order to permit site-specific conjugation of the PBD dimer with DAR2. This mutated antibody is referred to herein as AbA(S239C) and includes an AbA light chain and a modified AbA(C239) heavy chain sequence. The heavy chain amino acid sequence of AbA(S239C) is described below in SEQ ID NO: 1. The CDRs (CDR1, CDR2, and CDR3) (SEQ ID Nos: 3, 4, and 5, respectively) are underlined, and the variable region (SEQ ID NO: 2) is italicized.

(SEQ ID NO: 1)
EVQLQESGPGLVKPSQTLSLTCTVSGYSISRDFAWNWIRQPPGKGLEW
MGYISYNGNTRYQPSLKSRITISRDTSKNQFFLKLNSVTAADTATYYC
VTASRGFPYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPCVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALH
NHYTQKSLSLSPGK

The heavy chain constant region of AbA (S239C) contains a modified residue relative to its parent antibody AbA. Specifically, residue 239 (Kabat numbering) was mutated from S to a C relative to the heavy chain of AbA. This residue is underlined/bolded in SEQ ID NO: 1 above. It should be noted that S239C (Kabat numbering) corresponds to amino acid residue 238 of SEQ ID NO: 1 (S238C).

The light chain amino acid sequence (SEQ ID NO: 6) of AbA (S239C) is provided below, where CDR1, CDR2, and CDR3 (SEQ ID Nos. 8, 9, and 10, respectively) are underlined, and the variable region (SEQ ID NO: 7) is italicized.

(SEQ ID NO: 6)
DIQMTQSPSSMSVSVGDRVTITCHSSQDINSNIGWLQQKPGKSFKGLI
YHGTNLDDGVPSRFSGSGSGTDYTLTISSLQPEDFATYYCVQYAQFPW
TFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC

AbA (S239C) was further conjugated to a PBD dimer and tested as an ADC, as described in the examples below.

Ab1 was also modified in order to engineer site-specific conjugation sites of the warhead PBD. Specifically, an engineered cysteine antibody (C239) was generated using common techniques known in the art in order to permit site-specific conjugation of the PBD dimer with DAR 2. This mutated antibody is referred to herein as Ab1 (S239C) and includes an Ab1 light chain and a modified Ab1 (C239) heavy chain sequence. The heavy chain amino acid sequence of Ab1 (S239C) is described below in SEQ ID NO: 19. The CDRs (CDR1, CDR2, and CDR3) (SEQ ID NO: 16 to 18) are underlined, and the variable region (SEQ ID NO: 15) is italicized.

SEQ ID NO: 19
QVQLQESGPGLVKPSQTLSLTCTVSGYSISSDFAWNWIRQPPGKGLEW
MGYISYSGNTRYQPSLKSRITISRDTSKNQFFLKLNSVTAADTATYYC
VTAGRGFPYWGQGTLVTVSSASTKGPSVFPLAPSSKSTSGGTAALGCL
VKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPCVF
LFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNG
QPENNYKTTPPVLDSDGSFFLYSKLTVDKSRWQQGNVFSCSVMHEALH
NHYTQKSLSLSPGK

The heavy chain constant region of Ab1(S239C) contains a modified residue relative to its parent antibody. Specifically, residue 239 (Kabat numbering) was mutated from S to C relative to the heavy chain of Ab1. This residue is underlined/bolded in SEQ ID NO: 19 above. It should be noted that S239C (Kabat numbering) corresponds to amino acid residue 238 of SEQ ID NO: 19 (S238C).

Ab1 (S239C) was further conjugated to a PBD dimer and tested as an ADC, where Ab1(S239C)-PBD is comprised of two PBD drug-linker molecules conjugated to Cys engineered anti-EGFR antibody Ab1(S239C).

### Example 2: Generation and Physiochemical Characterization of PBD Conjugate

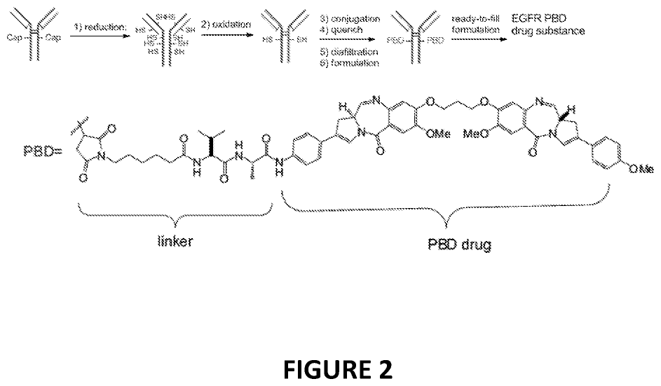

AbA (S239C)—PBD is comprised of two PBD drug-linker molecules conjugated to Cys engineered anti-EGFR antibody AbA(S239C). The structure of the PBD and linker are described in FIG. 2. FIG. 2 also describes the process by which AbA(S239C)-PBD was prepared. The conjugation process involved reducing the interchain disulfides, quantitative oxidation, and conjugation with excess PBD drug linker. The conjugation process consisted of a quantitative reduction of both the engineered and the interchain disulfides. The reduction mixture was then purified to remove the excess reagent and its byproducts, followed by quantitative oxidation of the interchain disulfides and then conjugation with excess PBD drug-linker. After quenching, the reaction mixture was purified and buffer-exchanged to yield AbA (S239C)—PBD with >85% DAR2 drug loading, as described in FIG. 2. The overall yield of the AbA (S239C)—PBD ADC after purification was approximately 90%. The conjugation process required the use of approximately 2.5% wt loading (˜2 g) of the PBD drug linker.

Ab1(S239C)-PBD, comprised of two PBD drug-linker molecules conjugated to Cys-engineered anti-EGFR antibody Ab1(S239C), was also prepared according to the process as described above and shown in FIG. 2.

### Example 3: Flow Cytometry Analysis

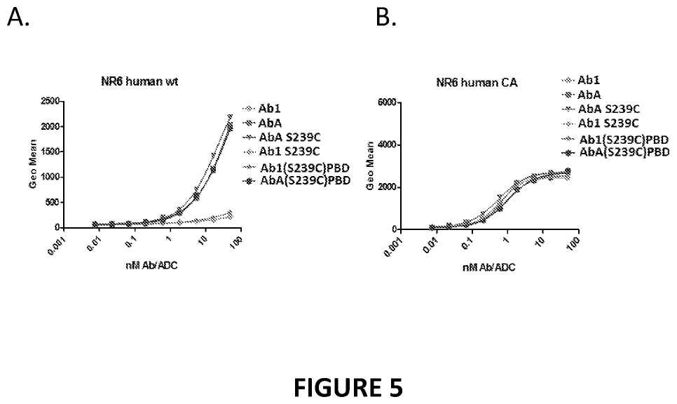

To confirm that the conjugation of Cys-engineered AbA(S239C) to PBD would not alter the binding properties compared to the parental antibody AbA, flow cytometry-based assays were performed with NR6 human fibroblast cells engineered to express wild-type EGFR or the CA mutant version of EGFR (EGFR^{C271A,C283A}), a point mutant known to expose the cryptic epitope recognized by Ab1 and AbA. Six antibodies/ADCs were included in the analysis, including Ab1 and AbA, the Cys-engineered Ab1(S239C) and AbA(S239C) mutants, and the PBD-conjugated Ab1(S239C)-PBD and AbA(S239C)-PBD. Increasing concentrations of antibodies were added to wild-type EGFR-overexpressing (FIG. 5A) and EGFR CA mutant-overexpressing (FIG. 5B) NR6 cells.

As shown in FIG. 5, binding curves with overexpressed EGFR are similar for AbA, AbA(S239C), and AbA(S239C)-PBD. Overall binding is greater with the CA mutant compared with the wild-type EGFR-expressing NR6 cells. All six antibodies/ADCs bound the EGFR CA mutant-expressing cells, as shown in FIG. 5B. These results indicate that the conjugation of Cys-engineered AbA(S239C) to PBD does not alter the binding properties compared to the parental antibody.

### Example 4: In Vitro Comparison of AbA(S239C)-PBD ADC Vs. AbA-MMAE ADC

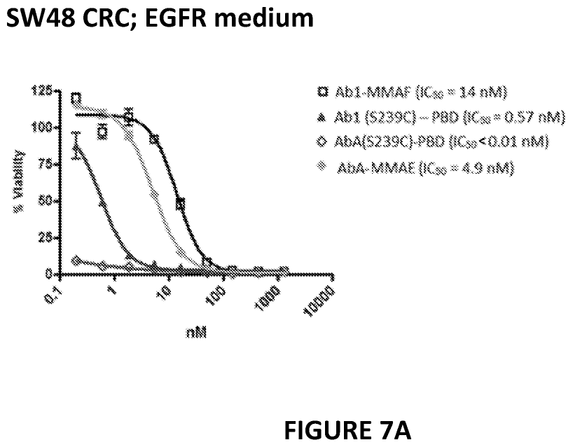

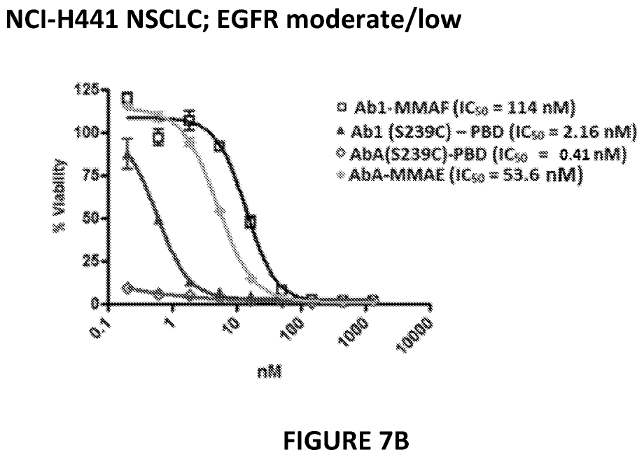

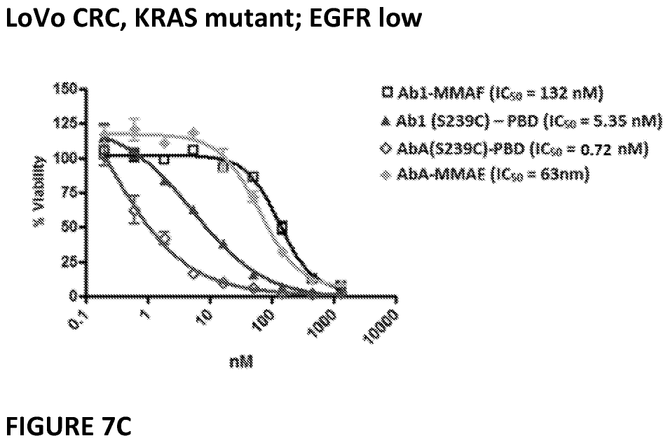

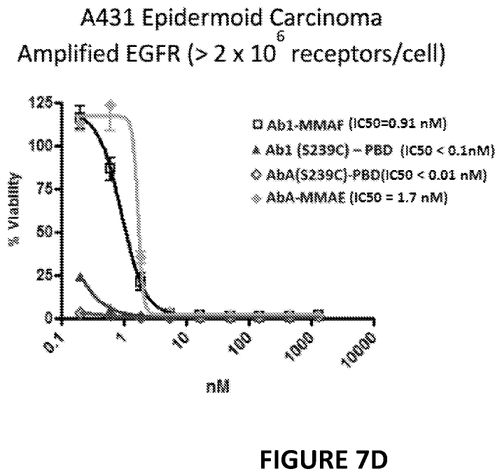

The cytotoxic activity of AbA(S239C)-PBD, along with Ab1(S239C)-PBD, was evaluated against a panel of tumor cell lines that express different levels of surface EGFR in cell killing assays. In particular A431, SW48, NC1-H441, and LoVo tumor cells were seeded in 96 well plates with ADCs (including AbA(S239C)-PBD and AbA-MMAE) added at the concentrations shown. Cell viability was assessed with the ATPlite Luminescence Assay after 72 hour incubation at 37° C. The results of this analysis are shown in FIG. 7. As shown in FIG. 7, there was improved cytotoxic activity in all four cell lines following treatment of the PBD conjugate AbA(S239C)-PBD compared to a corresponding auristatin conjugate, AbA-MMAE ADC.

For purposes of this disclosure, “AbA-MMAE” or (“AbA-vcMMAE”) refers to an auristatin based ADC, comprising AbA conjugated to the auristatin warhead monomethyl auristatin E via a cleavable valine-citrulline (VC) linker. It should be noted that, unless otherwise described, the AbA-MMAE ADC used in the Examples of the present disclosure has a DAR of about 3. For purposes of this disclosure, “Ab1-MMAF” refers to an antibody-drug conjugate (ADC) with the humanized IgG1 antibody Ab1 conjugated to the auristatin warhead monomethyl auristatin F via a noncleavable maleimidocaproyl linker. It should be noted that, unless otherwise described, the Ab1-MMAF ADC used in the Examples of the present disclosure has a DAR of about 3.8.

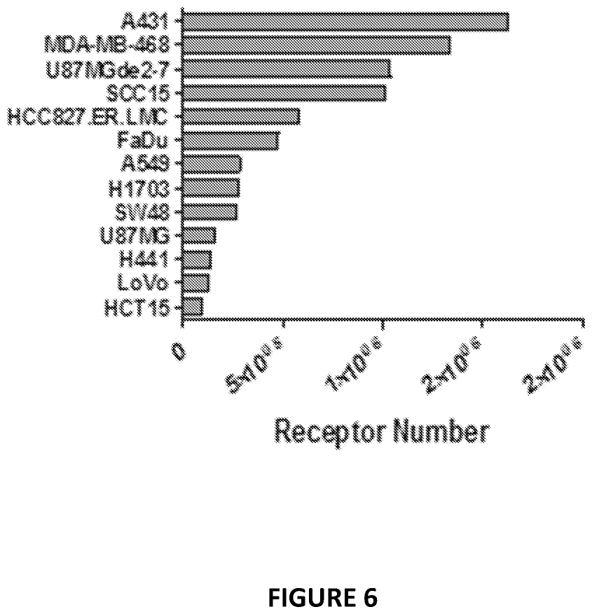

The EGFR number on the cells used in this analysis is shown in comparison to a number of other EGFR-overexpressing cell lines in FIG. 6. A431 is an epidermoid carcinoma cell line with amplified EGFR (>2×10⁶ receptors/cell). SW-48 is a colorectal adenocarcinoma cell line that expresses EGFR (>200,000 receptors per cell; IHC H-score 228); NCI-H441 is a lung adenoma xenograft model with moderate to low EGFR expression (˜100,000 receptors per cell; IHC H-score 150) and LoVo is a KRAS mutant colorectal adenocarcinoma with lower EGFR expression (<100,000 receptors per cell; IHC H-score 140) (FIG. 6).

AbA(S239C)-PBD and Ab1(S239C)-PBD were also evaluated for their ability to inhibit the growth of a panel of 22 colorectal cancer cell lines expressing different levels of EGFR (Table 1). Sensitivity to the ADCs, along with auristatin ADCs Ab1-MMAF and AbA-MMAE, in the cell proliferation assay is indicated by IC50 values. The AbA(S239C)-PBD and Ab1(S239C)-PBD conjugates used in this study contain >85% DAR 2 drug loading.

TABLE 1
Colorectal Cancer Cell Line EGFR Expression and Proliferation Assay Summary
with Ab1-MMAF ADC, AbA-MMAE ADC, AbA(S239C)-PBD, and Ab1(S239C)-PBD

| | RNA Linear | ADC and Free Drug IC50s (nM) | | | | |
|---|---|---|---|---|---|---|
| | Expression* | Ab1-MMAF | AbA-MMAE | Ab1(S239C)- | AbA(S239C)- | |
| CRC Line | EGFR | ADC | ADC | PBD ADC | PBD ADC | Ab095 PBD |
| WIDr | 15.6 | >133 | 90.6 | 6.9 | 5.5 | 34.8 |
| Colo320 HSR | 15.17 | >133 | >133 | 28.7 | 24.9 | >133 |
| SW1463 | 13.9 | >133 | >133 | 15.1 | 4.8 | 10.3 |
| Colo 201 | 9.61 | >133 | 81.6 | 5.1 | 2.6 | 19 |
| LS174T | 9.42 | >133 | >133 | 1.4 | 1 | 4.6 |
| Colo320 DM | 8.93 | >133 | >133 | 19.8 | 17.6 | 58.1 |
| T84 | 7.7 | >133 | >133 | 26.4 | 11.8 | 53 |
| HCT-15 | 7.22 | >133 | >133 | 38.8 | 28.4 | >133 |
| SW620 | 6.33 | >133 | 106 | 4.5 | 4.3 | 21.3 |
| RKO | 6.02 | >133 | 75.3 | 5.8 | 4.7 | 28.1 |
| LS1034 | 5.76 | >133 | >133 | 18.3 | 9.5 | 43.6 |
| SW48 | 4.49 | 26.3 | 8.6 | 0.23 | 0.015 | 1.9 |
| SW1116 | 3.65 | >133 | >133 | 10.8 | 4.6 | 37.4 |
| SW403 | 3.19 | >133 | 61.8 | 3.1 | 1.8 | 11.4 |
| HCT-116 | 2.03 | >133 | >133 | 9.4 | 12.0 | 42.6 |
| SW480 | 1.26 | >133 | >133 | 11.3 | 8 | 56.4 |
| LoVo | 1.24 | 132 | 63 | 5.35 | 0.72 | NT** |
| SK-CO-1 | 0.97 | >133 | 23.9 | 2.55 | 1.06 | 9.85 |
| CaCO2 | 0.8 | >133 | >133 | 19.6 | 13.9 | 41.1 |
| HT-29 | 0.77 | >133 | 56 | 4.86 | 1.54 | 26.2 |
| Colo 205 | 0.71 | >133 | >133 | 4.7 | 3.3 | 27.4 |
| DLD-1 | 0.7 | >133 | >133 | 9.2 | 9 | 21.3 |

*RNA determination by microarray analysis and presented as a linear value (from Oncomine).
**Not tested.

Microtubulin inhibitors have not demonstrated significant efficacy in some disease settings including EGFR-positive colorectal tumors. See Perez E A. Microtubule Inhibitors: Differentiating tubulin-inhibiting agents based on mechanisms of action, clinical activity, and resistance. Mol Cancer Ther 2009; 8(8): 2086-95. IHC analysis indicates that >25% of CRCs express EGFR, and CRC is an approved indication for several EGFR-based therapies. See Mendelsohn J, Baselga J. Epidermal growth factor receptor targeting in cancer. Semin Oncol 2006; 33(4):369-85; Herbst R S, Kim E S, Harari P M. IMC-C225, an anti-epidermal growth factor receptor monoclonal antibody, for treatment of head and neck cancer. Expert Opin Biol Ther 2001; 1(4):719-32; Lynch D H, Yang X D. Therapeutic potential of ABX-EGF: a fully human anti-epidermal growth factor receptor monoclonal antibody for cancer treatment. Semin Oncol 2002; 29(1 Suppl 4):47-50.

As shown in Table 1, whereas most of the cell lines were largely insensitive to the auristatin-based ADCs (AbA-MMAE ADC and Ab1-MMAF ADC), as evidenced by IC50 values generally >100 nm, the PBD conjugates AbA(S239C)-PBD and Ab1(S239c)-PBD were much more effective at inhibiting cell growth. Moreover, importantly, the inhibition of cell growth did not correlate with EGFR expression levels, suggesting that the PBD ADCs of the present disclosure can be effective against low EGFR expressing colorectal tumor cell lines. It is also possible that EGF ligand-induced autocrine activation and corresponding increased exposure of the AbA epitope may contribute to the sensitivity of some of these tumor cell lines. The non-targeting PBD ADC control also had some inhibitory activity against select tumor cell lines, but overall the activity was significantly reduced compared to that observed with the EGFR-targeting PBDs. In summary, these results indicate that the activity of the EGFR-PBD ADC may extend to low-level and mid-level EGFR-expressing colorectal tumors which are largely insensitive to auristatin-based ADCs.

The activity of the EGFR-PBD ADCs AbA(S239C)-PBD and Ab1(S239C)-PBD was also evaluated against a panel of human glioblastoma (GBM) tumor cell lines.

TABLE 2
Brain Cancer Cell Line EGFR Expression and Proliferation Assay Summary with
ABT-414, ABBV-221, ABT-806 PBD and AM-1 PBD ADCs

| | | | | AB1(S239C)- | AbA(S239C)- | |
|---|---|---|---|---|---|---|
| Brain | | Ab1-MMAF | AbA-MMAE | PBD | PBD | Ab095 |
| Tumor | EGFR* | ADC | ADC | ADC | ADC | PBD |
| U87MGde2-7 | >1.9 | 0.03 | 0.06 | 0.34 | 0.23 | >133 |
| A172 | 1.71 | 84.7 | 59.2 | 20.9 | 23.2 | 36.4 |
| T98G | 1.65 | >133 | 28.1 | 39.1 | 14.5 | >133 |
| MO59J | 1.55 | >133 | >133 | 6.8 | 2.8 | 26 |
| MO59K | 1.43 | >133 | >133 | 3.5 | 1.5 | 12.2 |
| LN-18 | 1.38 | >133 | NT** | 11 | 8.4 | 20 |
| SF264 | 1.33 | >133 | >133 | 5.3 | 1.4 | 24.3 |
| SF539 | 1.2 | >133 | >133 | 7.8 | 2.5 | 50.5 |
| SNB-19 | 1.15 | >133 | 75 | 7.2 | 3.7 | 39.2 |
| DBTRG- | 1.05 | >133 | >133 | 31.1 | 18 | 86.5 |
| 05MG | | | | | | |
| U87MG | 1 | 42.5 | 29.3 | 11.8 | 4.6 | 32.9 |
| U251 | 0.86 | >133 | 123 | 2.32 | 0.72 | 9.2 |
| U138MG | 0.79 | 20 | 14.5 | 16.5 | 16.1 | 23.3 |
| SNB-75 | 0.49 | >133 | >133 | 14.2 | 10.6 | 42.6 |
| CHLA-03- | 0.46 | >133 | >133 | 6 | 4.8 | 14.8 |
| AA | | | | | | |
| PFSK-1 | 0.01 | 66.2 | 58.6 | 1.6 | 1.9 | 4.64 |

*Protein expression was determined by Western blot analysis with an anti-EGFR antibody and normalized to U87MG
** Not tested

As indicated in Table 2, AbA-MMAE and Ab-1 MMAF were largely ineffective at inhibiting the proliferation of these tumor cell lines, as the cell lines included in this panel do not have amplified EGFR (with the exception of U87MGde2-7). Despite the lower levels of EGFR expressed on these tumor cell lines, the PBD conjugates AbA(S239C)-PBD and Ab1(S239C)-PBD had improved potency, consistent with the finding that the EGFR-PBD conjugates may be active in GBM beyond EGFR-amplified or overexpressed tumors.

### Example 5: In Vivo Characterization of EGFR ADCs

In vivo studies using xenograft models with EGFR expression levels varying from low to high in mouse subjects were performed using both AbA conjugated auristatin payloads and PBD payloads.

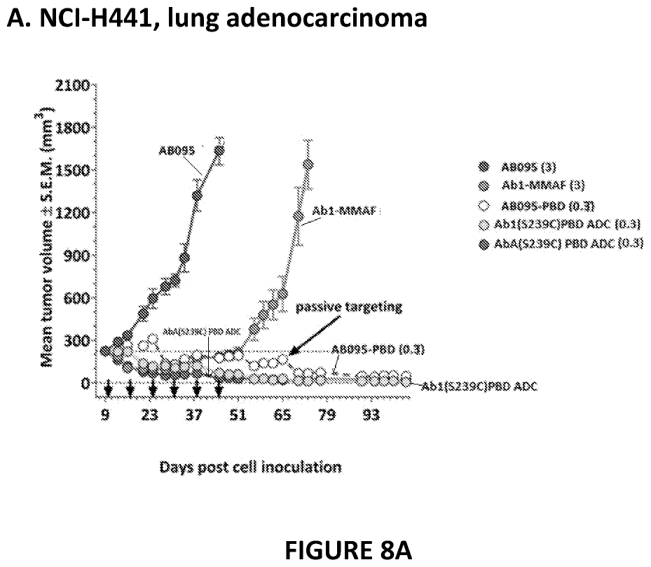

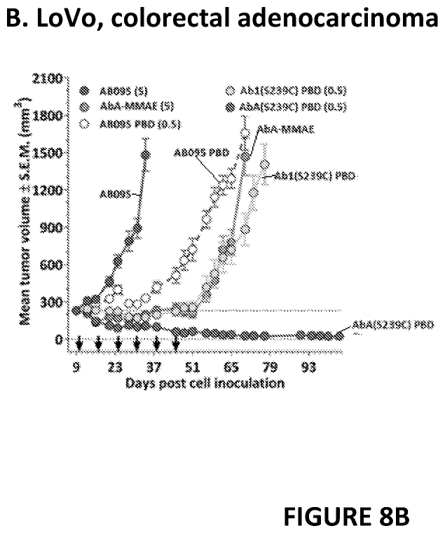

NCI-H441 is a lung adenoma xenograft model with moderate to low EGFR expression, as shown in FIG. 6 (˜100,000 receptors per cell, IHC H-score 150). The efficacy of AbA(S239C)-PBD and Ab1(S239C)-PBD in NCI-H441 (lung adenocarcinoma) is shown in FIG. 8A. As shown in FIG. 8A, AbA(S239C)-PBD and Ab1(S239C)-PBD, administered at 0.3 mg/kg once every seven days for a total of six doses (Q7D×6), induced complete and durable regressions in 100% of animals, whereas Ab1-MMAF administered at 10-fold higher doses (3 mg/kg) Q7D×6 induced complete responses in only 40% of the animals. A complete response (CR) is defined as tumor volume less than 25 mm³ for at least three consecutive measurements. All tumors eventually relapsed following Ab1-MMAF treatment. The negative control ADC, Ab095-PBD, also induced durable and complete responses in 100% of the animals. This sensitivity, observed with other ADCs, may result from the enhanced permeability and retention effect from a combination of PBD sensitivity and antibody accumulation in the NCI-H441 tumor rather than a recognition of the tumor-associated antigen. According to IHC, the expression of EGFR on the cell membranes of the NCI-H441 tumor cells was 3⁺.

FIG. 8B shows efficacy AbA(S239C)-PBD and of Ab1(S239C)-PBD in the colorectal adenocarcinoma LoVo xenograft. LoVo is a KRAS mutant colorectal adenocarcinoma with lower EGFR expression than NCI-H441 (<100,000 receptors per cell, IHC H-score 140). In the colorectal adenocarcinoma model, LoVo with lower target expression than NCI-H441, AbA(S239C)-PBD induced complete and durable responses, while tumors relapsed following cessation of dosing with Ab1(S239C)-PBD (FIG. 8B). Both conjugates were administered at 0.5 mg/kg on a q7d×6 regimen (where mice were dosed every 7 days for 6 weeks). In this model, specificity of the anti-EGFR conjugates was demonstrated by the increased durability of response compared to the negative control conjugate Ab095 PBD. AbA-MMAE was also active in this model, with activity similar to that observed with Ab1(S239C)-PBD, but not as active as AbA(S239C)-PBD. Further, in order to achieve these results, AbA-MMAE had to be administered at a much higher dose (specifically, at a 10-fold higher dose) than AbA(S239C)-PBD. In FIG. 8A and FIG. 8B, numbers in parenthesis represent dose in mg/kg. Arrows represent days of dosing. According to HC, the expression of EGFR on the cell membranes of LoVo tumor cells is 3+.

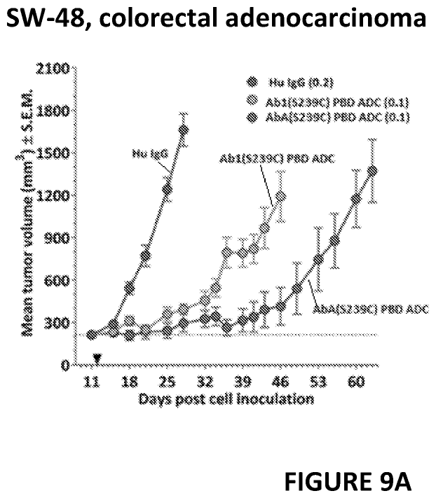

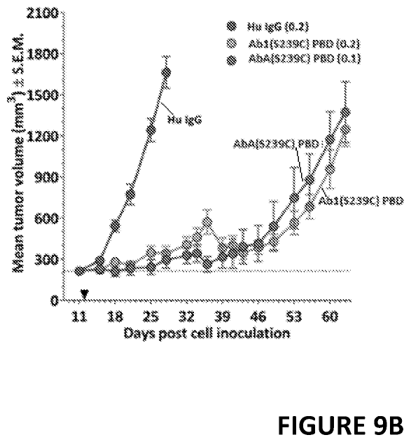

The efficacies of AbA(S239C)-PBD and Ab1(S239C)-PBD were assessed as compared to the corresponding auristatin ADCs in a second model of colorectal adenocarcinoma, SW48 (>200,000 receptors per cell; EGFR H-score: 228). Following a single dose of 0.1 mg/kg, AbA(S239C)-PBD induced a more durable response than Ab1(S239C)-PBD, as shown in FIG. 9A. The durability of response following Ab1(S239C)-PBD following dosing at 0.2 mg/kg was similar to that observed with AbA(S239C)-PBD at 0.1 mg/kg, suggesting that in this model, AbA(S239C)-PBD is at least two-fold more potent than Ab1(S239C)-PBD, as shown in FIG. 9B. In FIG. 9A and FIG. 9B, numbers in parenthesis represent dose in mg/kg. Arrows represent days of dosing. Expression of EGFR in SW48 xenografts as determined by IHC is 3+.

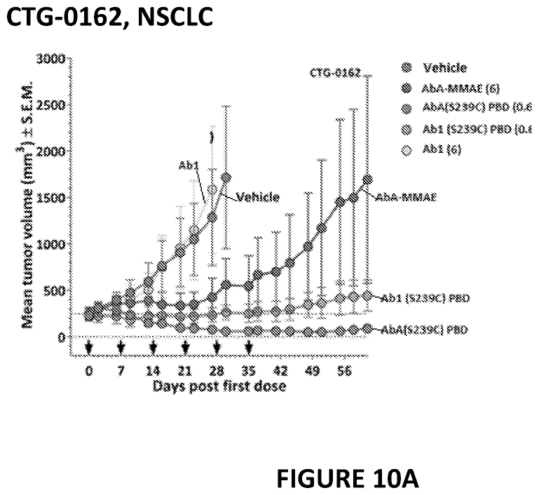

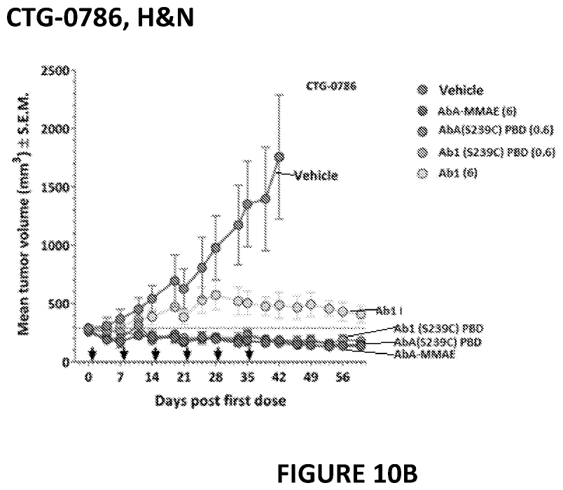

The efficacy of AbA(S239C)-PBD and Ab1(S239C)-PBD was also assessed relative to Ab1 and AbA-MMAE in the CTG-0162 non-small cell lung cancer model. As shown in FIG. 10A, in the CTG-0162 NSCLC model, AbA(S239C)-PBD and Ab1(S239C)-PBD dosed at q7×6 were very effective in inhibiting tumor growth, whereas AbA-MMAE was less efficacious, even though it was dosed ten-fold higher than AbA(S239C)-PBD or Ab1(S239C)-PBD. Ab1 was also not efficacious in this model.

The efficacy of AbA(S239C)-PBD and Ab1(S239C)-PBD was also assessed relative to Ab1 and AbA-MMAE in the CTG-9786 head and neck cancer model. As shown in FIG. 10B, in the CTG-9786 head and neck cancer model, Ab1(S239C)-PBD and AbA(S239C)-PBD dosed q7×6 were very effective at inhibiting tumor growth. AbA-MMAE was also effective, but required a much higher dose.

In summary, these in vivo results indicate that the PBD conjugates are more potent and produce more sustained anti-tumor responses than the auristatin-based conjugates across a variety of different tumor types, including lower EGFR-expressing colorectal tumors. Treatment of NSCLC, CRC, and H&N xenografts with AbA(S239C)-PBD significantly reduced tumor growth. The amplitude and durability of xenograft inhibition by AbA(S239C)-PBD was increased as compared to an auristatin conjugate where tested, and usually at a tenth of the dose of the auristatin conjugate at the same regimen.

### Example 6: In Vitro Plasma Stability

The stability of fluorescently labeled AbA(S239C) antibody and AbA(S239C)-PBD DAR2 was evaluated in vitro at 37° C. for 6 days in plasma from mouse, rat, cyno, and human, as well as in buffer. Protein aggregation and fragmentation were measured by size exclusion chromatography (SEC). Unconjugated PBD was determined by liquid chromatography-mass spectrometry (LC/MS/MS).

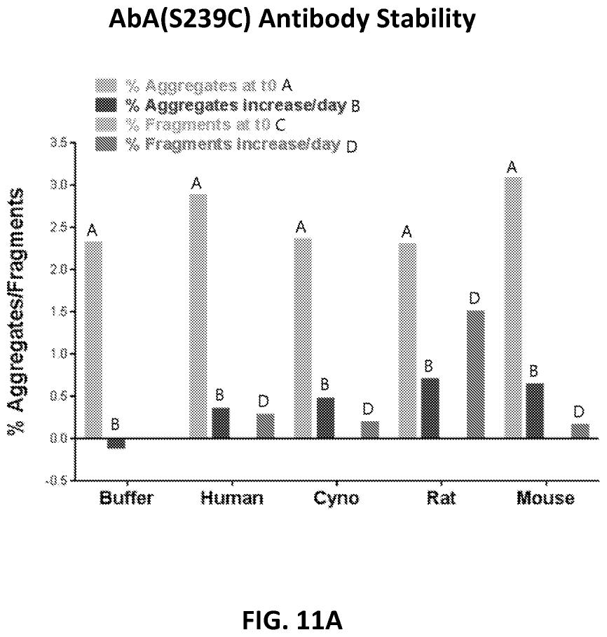

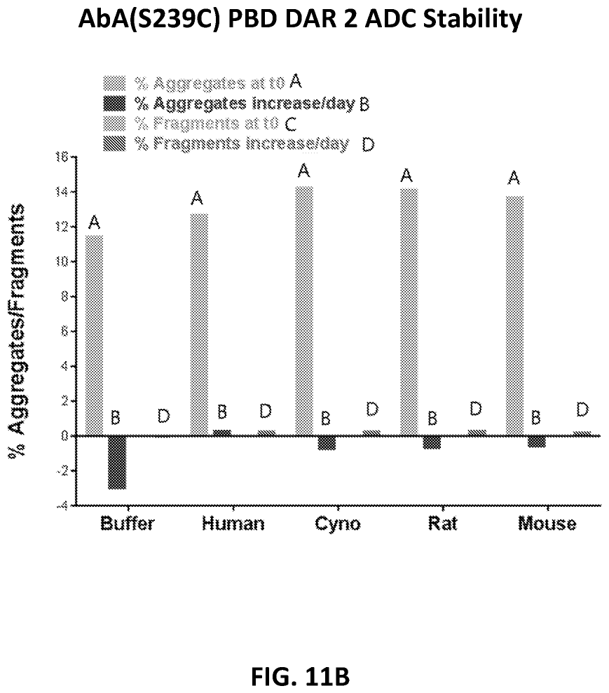

The in vitro plasma stability of the AbA(S239C) monoclonal antibody is shown in FIG. FIG. 11A. AbA(S239C) monoclonal antibody showed 2.3-3.1% initial aggregates at t0 in buffer and plasma with a low increase/day of aggregates (<0.7%) in buffer and plasma. The AbA(S239C) antibody had 0% initial fragments in buffer and plasma at to, and low increase per day of fragments (<1.5%) in buffer and plasma.

The in vitro plasma stability of the AbA(S239C) PBD DAR2 ADC is shown in FIG. FIG. 11B. AbA(S239C) PBD DAR 2 ADC showed high initial aggregates (11-13%) in buffer and plasma, and the % aggregates increase/day was either low (<0.3%) or decreased in buffer and plasma. The AbA(S239C) PBD DAR 2 ADC had 0% initial fragments in buffer and plasma, and minimal % increase/day (<0.3%) in buffer and plasma.

The PBD warhead itself was tested and found to be stable in plasma at 37° C. for 6 days in all plasma matrices. The unconjugated warhead released from the AbA(S239C) PBD DAR2 ADC was below the level of quantitation at all time points and in all matrices. This corresponds to <0.5% of the warhead equivalent dosed.

The stability of fluorescently labeled AbA-MMAE was also evaluated in vitro at 37° C. for 6 days in plasma (human, cyno, mouse, rat) and buffer. Protein aggregation and fragmentation were measured by size exclusion chromatography (SEC). AbA MMAE ADC showed 1.8-4.1% initial aggregates in buffer and plasma, with a % aggregates increase per day of 3.1-6.2% in plasma. The AbA MMAE ADC had 0-1.2% initial fragments in buffer and plasma at t0, and showed an increase per day of fragments of <1.4% in buffer and plasma.

Overall, despite having high initial aggregates, the in vitro plasma stability of the AbA(S239C) PBD DAR2 ADC was similar to (if not better than) AbA-MMAE ADC.

TABLE 3
ANTIBODY SEQUENCE TABLE

| SEQ | | |
|---|---|---|
| ID NO | Description | Sequence |
| 1 | AbA(S239C) heavy chain | EVQLQESGPGLVKPSQTLSLTCTVSGYSISRDFAWN |
| | (HC) | WIRQPPGKGLEWMGYISYNGNTRYQPSLKSRITISRD |
| | | TSKNQFFLKLNSVTAADTATYYCVTASRGFPYWGQ |
| | | GTLVTVSSASTKGPSVFPLAPSSKSTSGGTAALGCLV |
| | | KDYFPEPVTVSWNSGALTSGVHTFPAVLQSSGLYSL |
| | | SSVVTVPSSSLGTQTYICNVNHKPSNTKVDKKVEPK |
| | | SCDKTHTCPPCPAPELLGGPCVFLFPPKPKDTLMISR |
| | | TPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSREEMTKN |
| | | QVSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 2 | AbA HC variable region | EVQLQESGPGLVKPSQTLSLTCTVSGYSISRDFAWN |
| | | WIRQPPGKGLEWMGYISYNGNTRYQPSLKSRITISRD |
| | | TSKNQFFLKLNSVTAADTATYYCVTASRGFPYWGQ |
| | | GTLVTVSS |
| 3 | AbA HC CDR1 | GYSISRDFAWN |
| 4 | AbA HC CDR2 | YISYNGNTRYQPSLKS |
| 5 | AbA HC CDR3 | ASRGFPY |
| 6 | AbA light chain (LC) | DIQMTQSPSSMSVSVGDRVTITCHSSQDINSNIGWLQ |
| | NOTE: AbA and | QKPGKSFKGLIYHGTNLDDGVPSRFSGSGSGTDYTL |
| | AbA(S239C) have the | TISSLQPEDFATYYCVQYAQFPWTFGGGTKLEIKRTV |
| | same LC sequence | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 7 | AbA LC variable region | DIQMTQSPSSMSVSVGDRVTITCHSSQDINSNIGWLQ |
| | | QKPGKSFKGLIYHGTNLDDGVPSRFSGSGSGTDYTL |
| | | TISSLQPEDFATYYCVQYAQFPWTFGGGTKLEIK |
| 8 | AbA LC CDR1 | HSSQDINSNIG |
| 9 | AbA LC CDR2 | HGTNLDD |
| 10 | AbA LC CDR3 | VQYAQFPWT |
| 11 | AbA Heavy Chain (HC) | EVQLQESGPGLVKPSQTLSLTCTVSGYSISRDFAWN |
| | | WIRQPPGKGLEWMGYISYNGNTRYQPSLKSRITISRD |
| | | TSKNQFFLKLNSVTAADTATYYCVTASRGFPYWGQ |
| | | GTLVTVSSASTKGPSVFPLAPSSKSTSGGTAALGCLV |
| | | KDYFPEPVTVSWNSGALTSGVHTFPAVLQSSGLYSL |
| | | SSVVTVPSSSLGTQTYICNVNHKPSNTKVDKKVEPK |
| | | SCDKTHTCPPCPAPELLGGPSVFLFPPKPKDTLMISRT |
| | | PEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVYTLPPSREEMTKNQ |
| | | VSLTCLVKGFYPSDIAVEWESNGQPENNYKTTPPVL |
| | | DSDGSFFLYSKLTVDKSRWQQGNVFSCSVMHEALH |
| | | NHYTQKSLSLSPGK |

Note: AbA has the same HC sequence as AbA(S239C), but with a ser at position 239 (Kabat numbering); see SEQ ID NO: 11.

All publications, patents, patent applications, and other documents cited in this application are hereby incorporated by reference in their entireties for all purposes to the same extent as if each individual publication, patent, patent application, or other document were individually indicated to be incorporated by reference for all purposes.

While various specific embodiments have been illustrated and described, it will be appreciated that various changes can be made without departing from the spirit and scope of the present disclosure.

1. An antibody-drug conjugate (ADC) comprising the structure of Formula (X), or a salt thereof: 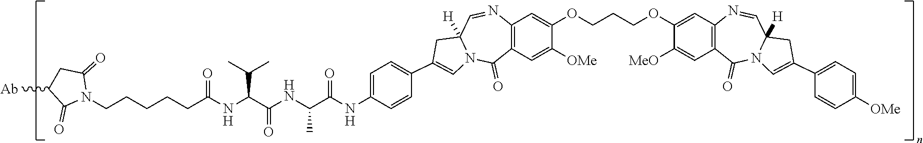
wherein Formula (X) comprises an anti-EGFR antibody (Ab) conjugated to a cytotoxic warhead,
wherein the anti-EGFR antibody comprises:
a heavy chain variable region comprising a CDRH1 sequence comprising SEQ ID NO: 3, a CDRH2 sequence comprising SEQ ID NO: 4, and a CDRH3 sequence comprising SEQ ID NO: 5;
a light chain variable region comprising a CDRL1 sequence comprising SEQ ID NO: 8, a CDRL2 sequence comprising SEQ ID NO: 9, and a CDRL3 sequence comprising SEQ ID NO: 10; and
a mutation comprising S239C in a heavy chain constant region, wherein the numbering is in accordance with Kabat;
wherein the anti-EGFR antibody is conjugated to the cytotoxic warhead through the mutation comprising S239C, and
wherein n is 2.

2. The ADC of claim 1, wherein the heavy chain variable region comprises SEQ ID NO: 2 and the light chain variable region comprises SEQ ID NO: 7.

3. The ADC of claim 1, comprising a full heavy chain comprising SEQ ID NO: 1, and a full light chain comprising SEQ ID NO: 6.

4. The ADC of claim 1, wherein the anti-EGFR antibody comprises an IgG1 isotype.

5. The ADC of claim 2, wherein the anti-EGFR antibody comprises an IgG1 isotype.

6. The ADC of claim 1, wherein the heavy chain constant region of the anti-EGFR antibody either lacks a C-terminal lysine or comprises an amino acid other than lysine at a C-terminus of the heavy chain constant region.

7. The ADC of claim 2, wherein the heavy chain constant region of the anti-EGFR antibody either lacks a C-terminal lysine or comprises an amino acid other than lysine at a C-terminus of the heavy chain constant region.

8. The ADC of claim 3, wherein the heavy chain constant region of the anti-EGFR antibody either lacks a C-terminal lysine or comprises an amino acid other than lysine at a C-terminus of the heavy chain constant region.

9. The ADC of claim 1, wherein the anti-EGFR antibody is a humanized antibody.

10. The ADC of claim 2, wherein the anti-EGFR antibody is a humanized antibody.

11. A pharmaceutical composition comprising the ADC of claim 1 in combination with at least one pharmaceutically acceptable excipient, carrier, or diluent.

12. The pharmaceutical composition of claim 11, wherein the drug-antibody ratio of the pharmaceutical composition is about 2.

13. An antibody-drug conjugate (ADC) comprising the structure of formula (IX), or a salt thereof, 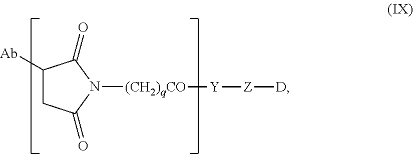
wherein D comprises a pyrrolobenzodiazepine (PBD) dimer; Ab is an anti-EGFR antibody, Y is Val, Z is Ala, and q is 1, 2, 3, 4, 5, 6, 7, or 8, and wherein the anti-EGFR antibody comprises
a heavy chain variable region comprising a CDRH1 sequence comprising SEQ ID NO: 3, a CDRH2 sequence comprising SEQ ID NO: 4, and a CDRH3 sequence comprising SEQ ID NO: 5;
a light chain variable region comprising a CDRL1 sequence comprising SEQ ID NO: 8, a CDRL2 sequence comprising SEQ ID NO: 9, and a CDRL3 sequence comprising SEQ ID NO: 10;
a mutation comprising S239C in a heavy chain constant region, wherein the numbering is in accordance with Kabat;
wherein the anti-EGFR antibody Ab is conjugated to the structure of Formula (IX) through the mutation comprising S239C, and
n is 2.

14. The ADC of claim 13, wherein q is 5.

15. The ADC of claim 13, wherein the heavy chain variable region comprises SEQ ID NO: 2, and the light chain variable region comprises SEQ ID NO: 7.

16. The ADC of claim 13, wherein the heavy chain comprises SEQ ID NO: 1, and the light chain comprises SEQ ID NO: 6.

17. The ADC of claim 13, wherein the anti-EGFR antibody comprises an IgG1 isotype.

18. The ADC of claim 14, wherein the heavy chain constant region of the anti-EGFR antibody either lacks a C-terminal lysine or comprises an amino acid other than lysine at a C-terminus of the heavy chain constant region.

19. A pharmaceutical composition comprising the ADC of claim 13 in combination with at least one pharmaceutically acceptable excipient, carrier, or diluent.

20. The pharmaceutical composition of claim 19, wherein the drug-antibody ratio of the pharmaceutical composition is about 2.


SEQUENCE LISTING (20 sequences)

SEQ ID NO: 1 - protein, 446 aa, Homo sapiens
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, Homo sapiens
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 3 - protein, 11 aa, Homo sapiens
GYSISRDFAWN

SEQ ID NO: 4 - protein, 16 aa, Homo sapiens
YISYNGNTRYQPSLKS

SEQ ID NO: 5 - protein, 7 aa, Homo sapiens
ASRGFPY

SEQ ID NO: 6 - protein, 214 aa, Homo sapiens
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 107 aa, Homo sapiens
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 8 - protein, 11 aa, Homo sapiens
HSSQDINSNIG

SEQ ID NO: 9 - protein, 7 aa, Homo sapiens
HGTNLDD

SEQ ID NO: 10 - protein, 9 aa, Homo sapiens
VQYAQFPWT

SEQ ID NO: 11 - protein, 446 aa, Homo sapiens
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTIVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPINPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 13 - protein, 645 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPERKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 14 - protein, 943 aa, Homo sapiens
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVALKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 15 - protein, 116 aa, Homo sapiens
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 16 - protein, 11 aa, Homo sapiens
GYSISSDFAWN

SEQ ID NO: 17 - protein, 16 aa, Homo sapiens
YISYSGNTRYQPSLKS

SEQ ID NO: 18 - protein, 7 aa, Homo sapiens
AGRGFPY

SEQ ID NO: 19 - protein, 446 aa, Homo sapiens
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 446 aa, Homo sapiens
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK